(19) United States
(12) Patent Application Publication
(10) Pub. No.: US 2025/0312275 A1
(43) Pub. Date: Oct. 9, 2025

(54) GAS PHASE DEPOSITION TECHNIQUE FOR PREPARATION OF PHARMACEUTICAL COMPOSITIONS

(71) Applicant: NANEXA AB, Uppsala (SE)

(72) Inventors: Erik LINDAHL, Uppsala (SE); Polla ROUF, Uppsala (SE); Mårten ROOTH, Uppsala (SE)

(21) Appl. No.: 18/873,160

(22) PCT Filed: Jun. 9, 2023
(86) PCT No.: PCT/GB2023/051512

§ 371 Date: Dec. 9, 2024

(30) Foreign Application Priority Data
Jun. 10, 2022 (GB) 2208517.9

(51) Int. Cl. A61K 9/00; A61K 31/53; A61K 33/30

(52) U.S. Cl. CPC A61K 9/0092; A61K 9/0021; A61K 31/53; A61K 33/30

(57) ABSTRACT

There is provided a process for the preparation of pharmaceutical or veterinary composition in the form of a plurality of particles, which process comprises: (a) loading a plurality of solid cores comprising a biologically-active agent into a stationary gas phase deposition reactor chamber, and (b) applying a gas phase deposition technique to surround, enclose and/or encapsulate said cores with one or more layers comprising one or more coating materials, each comprising one or more metal-containing or metalloid-containing compounds; and (c) sequentially repeating step (b) above as required to form a plurality of particles having a weight-, number-, and/or volume-based mean diameter that is between about 10 nm and about 100 μm, each particle comprising a respective solid core and a coating surrounding, enclosing and/or encapsulating said core, which gas phase deposition technique comprises: (1) introducing a pulse of a first reactant gas into the stationary gas phase deposition reactor chamber and allowing the first reactant gas to contact said solid cores for a pre-determined period of soaking time; (2) after step (1), evacuating and/or purging with an inert gas the stationary gas phase deposition reactor chamber; (3) introducing a pulse of a second reactant gas into the stationary gas phase deposition reactor chamber and allowing the second reactant gas to contact said solid cores for a pre-determined period of soaking time; and (4) after step (3), evacuating and/or purging with an inert gas the stationary gas phase deposition reactor chamber, wherein either the first reactant gas or the second reactant gas comprises a metal or metalloid-containing compound.

### FIELD OF THE INVENTION

This invention relates to a new process for the manufacture of compositions that are useful in the field of drug delivery.

### PRIOR ART AND BACKGROUND

The listing or discussion of an apparently prior-published document in this specification should not necessarily be taken as an acknowledgement that the document is part of the state of the art or common general knowledge.

In the field of drug delivery, the ability to control the profile of drug release is of critical importance. It is desirable to ensure that active ingredients are released at a desired and predictable rate in vivo following administration, in order to ensure a more optimal pharmacokinetic profile.

In the case of sustained release compositions, it is of critical importance that a drug delivery composition provides a release profile that shows minimal initial rapid release of active ingredient, that is a large concentration of drug in plasma shortly after administration. Such a ‘burst’ release may be hazardous in the case of drugs that have a narrow therapeutic window or drugs that are toxic at high plasma concentrations.

In the case of an injectable suspension of an active ingredient, it is also important that the size of the suspended particles is controlled so that they can be injected through a needle. If large, aggregated particles are present, they will not only block the needle, through which the suspension is to be injected, but also will not form a stable suspension within (i.e. they will instead tend to sink to the bottom of) the injection liquid.

There is, thus, a general need in the art for effective and/or improved drug transport and delivery systems.

Atomic layer deposition (ALD) is a technique that is employed to deposit thin films comprising a variety of materials, including organic, biological, polymeric and, especially, Inorganic materials, such as metal oxides, on solid substrates. It is an enabling technique for atomic and close-to-atomic scale manufacturing (ACSM) of materials, structures, devices and systems in versatile applications (see, for example, Zhang et al. Nanomanuf. Metrol. 2022, https://doi.org/10.1007/s41871-022-00136-8). Based on its self-limiting characteristics, ALD can achieve atomic-level thickness that is only controlled by adjusting the number of growth cycles. Moreover, multilayers can be deposited, and the properties of each layer can be customized at the atomic level.

Due to its atomic-level control, ALD is used as a key technique for the manufacturing of, for example, next-generation semiconductors, or in atomic-level synthesis of advanced catalysts as well as in the precise fabrication of nanostructures, nanoclusters, and single atoms (see, for example, Zhang et al. vide supra).

The technique is usually performed at low pressures and elevated temperatures. Film coatings are produced by alternating exposure of solid substrates within an ALD reactor chamber to vaporized reactants in the gas phase. Substrates can be silicon wafers, granular materials or small particles (e.g. microparticles or nanoparticles).

The coated substrate is protected from chemical reactions (decomposition) and physical changes by the solid coating. ALD can also potentially be used to control the rate of release of the substrate material within a solvent, which makes it of potential use in the formulation of active pharmaceutical ingredients.

In ALD, a first precursor, which can be metal-containing, is fed into an ALD reactor chamber (in a so called ‘precursor pulse’), and forms an adsorbed atomic or molecular monolayer at the surface of the substrate. Excess first precursor is then purged from the reactor, and then a second precursor, such as water, is pulsed into the reactor. This reacts with the first precursor, resulting in the formation of a monolayer of e.g. metal oxide on the substrate surface. A subsequent purging pulse is followed by a further pulse of the first precursor, and thus the start of a new cycle of the same events (a so called ‘ALD cycle’).

Alternatively, in ‘spatial ALD’, separate reactor chambers contain each precursor and the substrate being coated is moved from one reactor chamber to another in order for a coating to be formed. In this, or other methods of ALD, the introduction of a precursor to the substate being coated (or vice versa) may be considered equivalent to a ‘precursor pulse’ and the separation of a precursor from the substrate to be coated (or vice versa) may be considered equivalent to a ‘purging pulse’.

The thickness of the film coating is controlled by inter alia the number of ALD cycles that are conducted.

In a normal ALD process, because only atomic or molecular monolayers are produced during any one cycle, no discernible physical interface is formed between these monolayers, which essentially become a continuum at the surface of the substrate.

In international patent application WO 2014/187995, a process is described in which a number of ALD cycles are performed, which is followed by periodically removing the resultant coated substrates from the reactor and conducting a re-dispersion/agitation step to present new surfaces available for precursor adsorption.

The agitation step is done primarily to solve a problem observed for nano- and microparticles, namely that, during the ALD coating process, aggregation of particles takes place, resulting in ‘pinholes’ being formed by contact points between such particles. The re-dispersion/agitation step was performed by placing the coated substrates in water and sonicating, which resulted in deagglomeration, and the breaking up of contact points between individual particles of coated active substance.

The particles were then loaded back into the reactor and the steps of ALD coating of the powder, and deagglomerating the powder were repeated 3 times, to a total of 4 series of cycles. This process has been found to allow for the formation of coated particles that are, to a large extent, free of pinholes (see also, Hellrup et al., Int. J. Pharm., 529, 116 (2017)).

It has been found that the process of carrying out of ‘sets’ of ALD coating cycles followed by intermittent dispersion, as described in WO 2014/187995, results in clear, separate layers of coatings that are defined by clear, visible, physical interfaces between such coating layers. Such interfaces are more distinct than interfaces that can be seen between layers of different coating materials. The interfaces that form by such intermittent dispersion of the particles are clearly visible by a technique such as transmission electron microscopy (TEM) as regions of higher electron permeability. As explained below, similar interfaces are not visible when coatings of the same material are built up one atomic layer at a time from the surface of a substrate.

As described in international patent application WO 2021/111149, we have more recently found that it is advantageous to deagglomerate aggregated particles into primary particles externally to the reactor by a dry process that involves a combination of a mechanical forcing means and a sieve (in particular a sonic sifting device). This avoids the need for employing an aggressive deagglomeration technique such as sonication, as well as the need to dry particles prior to placing them back into the reactor for further coating. We have found that conducting the deagglomeration steps in this way allows for the presentation of essentially completely pinhole-free coated particles in a form that can be readily processed into a pharmaceutical formulation.

As described in unpublished UK patent application no. GB 2108305.0, we have even more recently found that it is advantageous to use a vibrational sieving technique to deagglomerate aggregated particles. In particular, the vibrational sieving technique results in the deagglomerated coated particles with the essential absence of cracks through which active ingredient can be released in an uncontrolled way.

In attempting to further scale up the processes described in WO 2021/111149 and UK patent application no. GB 2108305.0, we have found that the consistency of coatings between different particles coated together can become unsatisfactory as larger scale continuous flow reactors are used. This problem has been unexpectedly solved by way of the process described herein.

### DISCLOSURE OF THE INVENTION

According to a first aspect of the invention there is provided a process for the preparation of composition in the form of a plurality of particles, which process comprises:
(a) loading a plurality of solid cores comprising a biologically-active agent into a stationary gas phase deposition reactor chamber; and
(b) applying a gas phase deposition technique to surround, enclose and/or encapsulate said cores with one or more layers comprising one or more coating materials, each comprising one or more metal-containing or metalloid-containing compounds; and
(c) sequentially repeating step (b) above as required to form a plurality of particles having a weight-, number-, and/or volume-based mean diameter that is between about 10 nm and about 100 μm, each particle comprising a respective solid core and a coating surrounding, enclosing and/or encapsulating said core,
   which gas phase deposition technique comprises:
(1) introducing a pulse of a first reactant gas into the stationary gas phase deposition reactor chamber and allowing the first reactant gas to contact said solid cores for a pre-determined period of soaking time;
(2) after step (1), evacuating and/or purging with an inert gas the stationary gas phase deposition reactor chamber;
(3) introducing a pulse of a second reactant gas into the stationary gas phase deposition reactor chamber and allowing the second reactant gas to contact said solid cores for a pre-determined period of soaking time; and
(4) after step (3), evacuating and/or purging with an inert gas the stationary gas phase deposition reactor chamber,
   wherein either the first reactant gas or the second reactant gas comprises a metal or metalloid-containing compound.

In the process of the invention, both the first reactant gas and the second reactant gas, respectively, are allowed to contact the particle for a pre-determined period of soaking time. The term ‘soaking’ is used in this application only for clarity, that is, so that the relevant pre-determined period of time is readily distinguishable from other pre-determined periods of time that may be mentioned in the application. It is to be understood that the term ‘soaking’ is in no way limits the associated pre-determined period of time.

The process having such a soaking time increases the coating uniformity (also referred to as coating integrity or shell integrity, which may be measured for example as described hereinafter) because it allows each gas to diffuse conformally in high aspect-ratio substrates, e.g. especially powders. This is due to the substrates having an Increased surface area which needs a longer period of time to disuse and react with all of the available surface sites.

The aforementioned benefit of including a soaking time is even more pronounced when the process involves the use of reactants with slow reactivity because more time is provided for the reactant to react on the substrate surface. For example, this is evident especially for diethylzinc (DEZ) when depositing AlZnO as its reaction probability towards the surface is lower than for example trimethylaluminum (TMA).

Known ALD processes run in a continuous flow manner, meaning that a pump is actively pumping on the reactor during the whole process and gases pass continuously over the powder substrate. In contrast, including a soaking time in the process prevents the continuous flow of gases over the powder substrate. For example, introducing a soaking time may include closing a valve to prevent either inflow or outflow from the reactor chamber. If a valve is closed to prevent inflow (e.g. the valve is positioned to close an inlet to the reactor chamber), pressure in the reactor chamber may be substantially constant during the soaking time. However, some change in pressure may occur due to reactions taking place and the pressure change would depend on the stoichiometry of those reactions. Alternatively, if a valve is closed to prevent outflow (e.g. the valve is positioned to close an outlet of the reactor chamber), pressure in the reactor chamber may steadily increase during the soaking time as more precursor enters the reactor chamber. This may beneficially urge the precursor further into the powder bed and increase the likelihood of reaction with the surfaces of particles therein, thereby enhancing the effect of the soaking time.

An ALD process including such soaking times is sometimes referred to as a “stop-flow” process.

The inclusion of a soaking time will not only generate coatings with good shell integrity and more controlled release profiles but it also gives a coating composition closer towards the ALD process setup. When using an ALD cycle scheme consisting of three DEZ cycles and one TMA cycle one would expect to get an atomic ratio of 3:1 between Zn and Al in the resulting shell. This is not the case when depositing with continuous flow, where the atomic ratio nears 1:1 because of the lower reaction probability for DEZ than TMA. When using stop-flow the same ratio is very close to 3:1.

The process of the invention also requires using a stationary gas phase deposition reactor chamber. It will be understood that a stationary reactor chamber in the context of the invention is a reactor chamber that remains stationary while in use to perform a gas phase deposition technique, excluding negligible vibrations caused by associated machinery. This is in contrast to a reactor chamber which rotates or vibrates or otherwise actively moves during the gas phase deposition process.

It will be understood that the “pre-determined period” of soaking time may be any suitable period of time. The period of time is largely dependent on the particle sample size (or batch size), wherein the smaller the sample size the less soaking time is required whilst the larger the sample size the longer soaking time is required. The soaking time may also be dependent on factors such as the characteristics of the solid cores and/or the type of reactant gas. Additionally, the soaking time may depend on the design of the ALD reactor, e.g., size of the reaction chamber, distance to the inlet valves and the valve to the pump. The soaking time may be in range of about 2 seconds to about 30 minutes. For example, the soaking time may be about 30 seconds, 1 minute, 3 minutes, 10 minutes or 15 minutes.

It will be appreciated by a person skilled in the art that, for any combination of variables mentioned above, the beneficial effect provided by a soaking time is finite. For example, for a given process, a first 5 second period of a soaking time may provide a significant benefit to coating uniformity and integrity, a second 5 second period of soaking time may provide some additional benefit but less than that provided by the first period and a third 5 second period may be an almost negligible additional benefit over the benefit that had already been gained (this is a simplified example for demonstrative purposes only). In other words. It is predicted that the benefit provided by soaking time as the soaking time is increased can be modelled as a curve that increases with a high gradient initially but the gradient will reduce as the soaking time is increased until a steady state is eventually reached at which point further soaking may no longer be advantageous.

Although the ‘benefit’ of a soaking time is not limited to shell integrity, it will be appreciated that the shell integrity of resulting particles may be used as an indicator of the effectiveness of the soaking time. Accordingly, to select a suitable soaking time for a particular process, a person skilled in the art may run trials including incrementally increasing soaking times to determine a desirable compromise between the length of soaking time and the benefit it provides to shell integrity. In the simplified example referred to above, the skilled person may determine that a soaking time close to 10 seconds is appropriate as minimal additional benefit would be observed with any greater soaking time.

In some examples of the invention, the predetermined period of soaking time may be selected to provide a shell integrity, as measured for example as described hereinafter of at least about 70%, such as at least about 80%, such as at least about 90%, such as at least about 95%, such as about 98.5%. It is to be understood that shell or coating integrity, as described herein, may be considered as the inverse of API dissolution in a solvent which dissolves the API but not the coating material. For example, API dissolution of about 5% would be indicative of a shell/coating integrity of about 95%. Suitable methods for determining coating integrity via determination of API dissolution are provided in the examples described below.

During the process of the invention, the valve to the pump inlet is closed so that the reactant gas resides in the reaction chamber without any active pumping for the pre-determined soaking time before the chamber is then pumped. Depending on the reactor, the valve may be completely shut off so that no gas is passed through, or it may not be possible to completely shut off the valve such that there is the presence of minimal amount of pumping (e.g. a nitrogen carrier gas may still flow in the chamber), In either scenario, it is understood that no active pumping is occurring, That is to say, there is the substantial absence of pumping.

Moreover, the pre-determined period of soaking time is preferably carried out in the substantial absence of mechanical agitation of the plurality of solid cores. Agitation or sieving of the solid cores may take place during the other steps of the process, as described further on in this application.

The process may optionally include carrying out multipulses, i.e. short burst of in-flow, of the reactant gas without purging/rinsing in between each multipulse, with each multipulse pumping the reactor for a pre-determined period of pumping time. Such multipulsing is equivalent to carrying out a single long pulse of reactant gas in terms of the amount of reactant substrate that is applied to the solid cores. By applying the reactant in a multipulse manner, a more consistent coverage of the solid cores is achieved, Each multipulse pumping time may be from about 0.1 to 1000 seconds, about 1 to 500 seconds, about 2 to 250 seconds, about 3 to 100 seconds, about 4 to 50 seconds, or about 5 to 10 seconds, for example 9 seconds. The multipulses may be applied about 5 to 1000 times, about 10 to 250 times, or about 20 to 50 times in a single step.

The term ‘solid’ will be well understood by those skilled in the art to include any form of matter that retains its shape and density when not confined, and/or in which molecules are generally compressed as tightly as the repulsive forces among them will allow. The solid cores have at least a solid exterior surface onto which a layer of coating material can be deposited. The interior of the solid cores may be also solid or may instead be hollow. For example, if the particles are spray dried before they are placed into the reactor vessel, they may be hollow due to the spray drying technique. Cores may in the alternative comprise agglomerates of smaller ‘primary’ particles, i.e. secondary particles of a size range defined herein, which are subsequently coated as described herein.

The process of the invention is preferably employed to make pharmaceutical compositions. Accordingly, the composition, and in particular the solid cores, comprise a pharmacologically-effective amount of a biologically active agent.

In this respect, the solid cores may consist essentially of, or comprise, biologically active agent (which agent may hereinafter be referred to interchangeably as a ‘drug’, and ‘active pharmaceutical ingredient (API)’ and/or an ‘active ingredient’). Biologically active agents also include biopharmaceuticals and/or biologics. Biologically active agents can also include a mixture of different APIs, as different API particles or particles comprising more than one API.

By ‘consists essentially’ of biologically-active agent, we include that the solid core is essentially comprised only of biologically active agent(s), i.e. it is free from non-biologically active substances, such as excipients, carriers and the like (vide infra), and from other active substances. This means that the core may comprise less than about 5%, such as less than about 3%, including less than about 2%, e.g. less than about 1% of such other excipients and/or active substances.

In the alternative, cores comprising biologically active agents may include such an agent in admixture with one or more pharmaceutical ingredients, which may include pharmaceutically-acceptable excipients, such as adjuvants, diluents or carriers, and/or may include other biologically-active ingredients.

Biologically active agents may be presented in a crystalline, a part-crystalline and/or an amorphous state. Biologically active agents may further comprise any substance that is in the solid state, or which may be converted into the solid state, at about room temperature (e.g. about 18° C.) and about atmospheric pressure, irrespective of the physical form. Such agents (and optionally other pharmaceutical Ingredients as mentioned herein) should also remain in the form of a solid whilst being coated in, for example, an ALD reactor and also should not decompose physically or chemically to an appreciable degree (i.e. no more than about 10% w/w) whilst being coated, or after having been covered by at least one of the coating material. Biologically active agents may further be presented in combination (e.g. in admixture or as a complex) with another active substance.

As used herein, the term ‘biologically active agent’, or similar and/or related expressions, generally refer(s) to any agent, or drug, capable of producing some sort of physiological effect (whether in a therapeutic or prophylactic capacity against a particular disease state or condition) in a living subject, including, in particular, mammalian and especially human subjects (patients).

Biologically-active agents may, for example, be selected from an analgesic, an anaesthetic, an anti-ADHD agent, an anorectic agent, an antiaddictive agent, an antibacterial agent, an antimicrobial agent, an antifungal agent, an antiviral agent, an antiparasitic agent, an antiprotozoal agent, an anthelmintic, an ectoparasiticide, a vaccine, an anticancer agent, an antimetabolite, an alkylating agent, an antineoplastic agent, a topoisomerase inhibitor, an immunomodulator, an immunostimulant, an immunosuppressant, an anabolic steroid, an anticoagulant agent, an antiplatelet agent, an anticonvulsant agent, an antidementia agent, an antidepressant agent, an antidote, an antihyperlipidemic agent, an antigout agent, an antimalarial, an antimigraine agent, an antiparkinson agent, an antipruritic agent, an antipsoriatic agent, an antiemetic, an anti-obesity agent, an antiasthma agent, an antibiotic, an antidiabetic agent, an antiepileptic, an antifibrinolytic agent, an antihemorrhagic agent, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, an antiparkinsonian agent, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, an antidiarrheals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radio-pharmaceutical, a hormone, a sedative, an anti-allergic agent, an appetite stimulant, a steroid, a sympathomimetic, a thrombolytic, a thyroid agent, a vasodilator, a xanthine, an erectile dysfunction improvement agent, a gastrointestinal agent, a histamine receptor antagonist, a keratolytic, an antlanginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a radiopharmaceutical, a senotherapeutic, a vitamin, or a mixture of any of these.

The biologically-active agent may also be a cytokine, a peptidomimetic, a peptide, a protein, a toxoid, a serum, an antibody, a vaccine, a nucleoside, a nucleotide, a portion of genetic material, a nucleic acid, or a mixture thereof. Non-limiting examples of therapeutic peptides/proteins are as follows: lepirudin, cetuximab, dornase alfa, denileukin diftitox, etanercept, bivalirudin, leuprolide, alteplase, interferon alfa-n1, darbepoetin alfa, reteplase, epoetin alfa, salmon calcitonin, interferon alfa-n3, pegfilgrastim, sargramostim, secretin, peginterferon alfa-2b, asparaginase, thyrotropin alfa, antihemophilic factor, anakinra, gramicidin D, intravenous immunoglobulin, anistreplase, insulin (regular), tenecteplase, menotropins, interferon gamma-1b, interferon alfa-2a (recombinant), coagulation factor VIIa, oprelvekin, palifermin, glucagon (recombinant), aldesleukin, botulinum toxin Type B, omalizumab, lutropin alfa, insulin lispro, insulin glargine, collagenase, rasburicase, adalimumab, Imiglucerase, abciximab, alpha-1-proteinase inhibitor, pegaspargase, interferon beta-1a, pegademase bovine, human serum albumin, eptifibatide, serum albumin iodinated, infliximab, follitropin beta, vasopressin, interferon beta-1b, hyaluronidase, rituximab, basiliximab, muromonab, digoxin immune Fab (ovine), ibritumomab, daptomycin, tositumomab, pegvisomant, botulinum toxin type A, pancrelipase, streptokinase, alemtuzumab, alglucerase, capromab, laronidase, urofollitropin, efalizumab, serum albumin, choriogonadotropin alfa, antithymocyte globulin, filgrastim, coagulation factor IX, becaplermin, agalsidase beta, interferon alfa-2b, oxytocin, enfuvirtide, palivizumab, daclizumab, bevacizumab, arcitumomab, eculizumab, panitumumab, ranibizumab, idursulfase, alglucosidase alfa, exenatide, mecasermin, pramlintide, galsulfase, abatacept, cosyntropin, corticotropin, insulin aspart, insulin detemir, insulin glulisine, pegaptanib, nesiritide, thymalfasin, defibrotide, natural alpha interferon/multiferon, glatiramer acetate, preotact, teicoplanin, canakinumab, ipilimumab, sulodexide, tocilizumab, teriparatide, pertuzumab, rilonacept, denosumab, liraglutide, semaglutide, exenatide, lixisenatide, albiglutide, dulaglutide, tirzepatide, golimumab, belatacept, buserelin, velaglucerase alfa, tesamorelin, brentuximab vedotin, belimumab, aflibercept, taliglucerase alfa, asparaginase Erwinia chrysanthemi, ocriplasmin, glucarpidase, teduglutide, raxibacumab, certolizumab pegol, insulin isophane, epoetin zeta, obinutuzumab, fibrinolysin aka plasmin, follitropin alpha, romiplostim, lucinactant, natalizumab, aliskiren, ragweed pollen extract, secukinumab, somatotropin (recombinant), drotrecogin alfa, alefacept, OspA lipoprotein, urokinase, abarelix, sermorelin, aprotinin, gemtuzumab ozogamicin, satumomab pendetide, antithrombin alfa, antithrombin III (human), asfotase alfa, atezolizumab, autologous cultured chondrocytes, beractant, blinatumomab, C1 esterase inhibitor (human), coagulation factor XIII A-subunit (recombinant), conestat alfa, daratumumab, desirudin, elosulfase alfa, evolocumab, fibrinogen concentrate (human), filgrastim-sndz, gastric intrinsic factor, hepatitis B immune globulin, human calcitonin, human Clostridium tetani toxoid immune globulin, human rabies virus immune globulin, human Rho (D) immune globulin, human Rho (D) immune globulin, hyaluronidase (human, recombinant), idarucizumab, immune globulin (human), vedolizumab, ustekinumab, turoctocog alfa, tuberculin purified protein derivative, simoctocog alfa, siltuximab, sebelipase alfa, sacrosidase, ramucirumab, prothrombin complex concentrate, poractant alfa, pembrolizumab, peginterferon beta-1a, ofatumumab, obiltoxaximab, nivolumab, necitumumab, metreleptin, methoxy polyethylene glycol-epoetin beta, mepolizumab, ixekizumab, insulin degludec, insulin (porcine), insulin (bovine), thyroglobulin, anthrax immune globulin (human), anti-inhibitor coagulant complex, brodalumab, C1 esterase inhibitor (recombinant), chorionic gonadotropin (human), chorionic gonadotropin (recombinant), coagulation factor X (human), dinutuximab, efmoroctocog alfa, factor IX complex (human), hepatitis A vaccine, human varicella-zoster immune globulin, ibritumomab tiuxetan, lenograstim, pegloticase, protamine sulfate, protein S (human), sipuleucel-T, somatropin (recombinant), susoctocog alfa and thrombomodulin alfa, as well as sarcomeres and synthetic forms of antisense RNA, RNA interference agent, messenger RNA, transfer RNA, ribosomal RNA, including RNA aptameres.

Non-limiting examples of drugs which may be used according to the present invention are all-trans retinoic acid (tretinoin), alprazolam, allopurinol, amiodarone, amlodipine, asparaginase, astemizole, atenolol, azathioprine, azelatine, beclomethasone, bendamustine, bleomycin, budesonide, buprenorphine, butalbital, capecitabine, carbamazepine, carbidopa, carboplatin, cefotaxime, cephalexin, chlorambucil, cholestyramine, ciprofloxacin, cisapride, cisplatin, clarithromycin, clonazepam, clozapine, cyclophosphamide, cyclosporin, cytarabine, dacarbazine, dactinomycin, daunorubicin, diazepam, diclofenac sodium, digoxin, dipyridamole, divalproex, dobutamine, docetaxel, doxorubicin, doxazosin, enalapril, epirubicin, erlotinib, estradiol, etodolac, etoposide, everolimus, famotidine, felodipine, fentanyl citrate, fexofenadine, filgrastim, finasteride, fluconazole, flunisolide, fluorouracil, flurbiprofen, fluralaner, fluvoxamine, furosemide, gemcitabine, glipizide, gliburide, ibuprofen, ifosfamide, imatinib, indomethacin, irinotecan, isosorbide dinitrate, isotretinoin, isradipine, itraconazole, ketoconazole, ketoprofen, lamotrigine, lansoprazole, loperamide, loratadine, lorazepam, lovastatin, medroxyprogesterone, mefenamic acid, mercaptopurine, mesna, methotrexate, methylprednisolone, midazolam, mitomycin, mitoxantrone, moxidectine, mometasone, nabumetone, naproxen, nicergoline, nifedipine, norfloxacin, omeprazole, oxaliplatin, paclitaxel, phenyloin, piroxicam, procarbazine, quinapril, ramipril, risperidone, rituximab, sertraline, simvastatin, sulindac, sunitinib, temsirolimus, terbinafine, terfenadine, thioguanine, trastuzumab, triamcinolone, valproic acid, vinblastine, vincristine, vinorelbine, zolpidem, or pharmaceutically-acceptable salts of any of these.

Compositions made by the process of the invention may comprise benzodiazipines, such as alprazolam, chlordiazepoxide, clobazam, clorazepate, diazepam, estazolam, flurazepam, lorazepam, oxazepam, quazepam, temazepam, triazolam and pharmaceutically-acceptable salts of any of these.

Anaesthetics that may also be employed in the compositions made by the process of the invention may be local or general. Local anaesthetics that may be mentioned include amylocaine, ambucaine, articaine, benzocaine, benzonatate, bupivacaine, butacaine, butanilicaine, chloroprocaine, cinchocaine, cocaine, cyclomethycaine, dibucaine, diperodon, dimethocaine, eucaine, etidocaine, hexylcaine, fomocaine, fotocaine, hydroxyprocaine, isobucaine, levobupivacaine, lidocaine, mepivacaine, meprylcaine, metabutoxycaine, nitracaine, orthocaine, oxetacaine, oxybuprocaine, paraethoxycaine, phenacaine, piperocaine, piridocaine, pramocaine, prilocaine, primacaine, procaine, procainamide, proparacaine, propoxycaine, pyrrocaine, quinisocaine, ropivacaine, trimecaine, tolycaine, tropacocaine, or pharmaceutically-acceptable salts of any of these.

Psychiatric drugs may also be employed in the compositions made by the process of the invention. Psychiatric drugs that may be mentioned include 5-HTP, acamprosate, agomelatine, alimemazine, amfetamine, dexamfetamine, amisulpride, amitriptyline, amobarbital, amobarbital/secobarbital, amoxapine, amphetamine(s), aripiprazole, asenapine, atomoxetine, baclofen, benperidol, bromperidol, bupropion, buspirone, butobarbital, carbamazepine, chloral hydrate, chlorpromazine, chlorprothixene, citalopram, clomethiazole, clomipramine, clonidine, clozapine, cyclobarbital/diazepam, cyproheptadine, cytisine, desipramine, desvenlafaxine, dexamfetamine, dexmethylphenidate, diphenhydramine, disulfiram, divalproex sodium, doxepin, doxylamine, duloxetine, enanthate, escitalopram, eszopiclone, fluoxetine, flupenthixol, fluphenazine, fluspirilen, fluvoxamine, gabapentin, glutethimide, guanfacine, haloperidol, hydroxyzine, iloperidone, imipramine, lamotrigine, levetiracetam, levomepromazine, levomilnacipran, lisdexamfetamine, lithium salts, lurasidone, melatonin, melperone, meprobamate, metamfetamine, nethadone, methylphenidate, mianserin, mirtazapine, moclobemide, naltrexone, nalmefene, niaprazine, nortriptyline, olanzapine, ondansetron, oxcarbazepine, paliperidone, paroxetine, penfluridol, pentobarbital, perazine, pericyazine, perphenazine, phenelzine, phenobarbital, pimozide, pregabalin, promethazine, prothipendyl, protriptyline, quetiapine, ramelteon, reboxetine, reserpine, risperidone, rubidium chloride, secobarbital, selegiline, sertindole, sertraline, sodium oxybate, sodium valproate, sodium valproate, sulpiride, thioridazine, thiothixene, tianeptine, tizanidine, topiramate, tranylcypromine, trazodone, trifluoperazine, trimipramine, tryptophan, valerian, valproic acid in 2.3:1 ratio, varenicline, venlafaxine, vilazodone, vortioxetine, zaleplon, ziprasidone, zolpidem, zopicione, zotepine, zuclopenthixol and pharmaceutically-acceptable salts of any of these.

Antiparkinsonism drugs that may be mentioned include levodopa and apomorphine and pharmaceutically-acceptable salts of these.

Opioid analgesics that may be employed in compositions made by the process of the invention include buprenorphine, butorphanol, codeine, fentanyl, hydrocodone, hydromorphone, meperidine, methadone, morphine, nomethadone, opium, oxycodone, oxymorphone, pentazocine, tapentadol, tramadol and pharmaceutically-acceptable salts of any of these.

Opioid antagonists that may be employed in compositions made by the process of the invention include naloxone, nalorphine, niconalorphine, diprenorphine, levallorphan, samidorphan, nalodeine, alvimopan, methylnaltrexone, naloxegol, 6ß-naltrexol, axelopran, bevenopran, methylsamidorphan, naldemedine, preferably nalmefene and, especially, naltrexone, as well as pharmaceutically-acceptable salts of any of these.

Anticancer agents that may be included in compositions made by the process of the invention include the following: actinomycin, afatinib, all-trans retinoic acid, amsakrin, anagrelid, arseniktrioxid, axitinib, azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, dasatinib, daunorubicin, decitabine, docetaxel, doxifluridine, doxorubicin, epirubicin, epothilone, erlotinib, estramustin, etoposide, everolimus, fludarabine, fluorouracil, gefitinib, guadecitabine, gemcitabine, hydroxycarbamide, hydroxyurea, idarubicin, idelalisib, ifosfamide, imatinib, irinotecan, ixazomib, kabozantinib, karfilzomib, krizotinib, lapatinib, lomustin, mechlorethamine, melphalan, mercaptopurine, mesna, methotrexate, mitotan, mitoxantrone, nelarabin, nilotinib, niraparib, olaparib, oxaliplatin, paclitaxel, panobinostat, pazopanib, pemetrexed, pixantron, ponatinib, procarbazine, regorafenib, ruxolitinib, sonidegib, sorafenib, sunitinib, tegafur, temozolomid, teniposide, tioguanine, tiotepa, topotecan, trabektedin, valrubicin, vandetanib, vemurafenib, venetoklax, vinblastine, vincristine, vindesine, vinflunin, vinorelbine, vismodegib, as well as pharmaceutically-acceptable salts of any of these.

Such compounds may be used in any one of the following cancers: adenoid cystic carcinoma, adrenal gland cancer, amyloidosis, anal cancer, ataxia-telangiectasia, atypical mole syndrome, basal cell carcinoma, bile duct cancer, Birt-Hogg Dubé, tube syndrome, bladder cancer, bone cancer, brain tumor, breast cancer (Including breast cancer in men), carcinoid tumor, cervical cancer, colorectal cancer, ductal carcinoma, endometrial cancer, esophageal cancer, gastric cancer, gastrointestinal stromal tumor, HER2-positive, breast cancer, islet cell tumor, juvenile polyposis syndrome, kidney cancer, laryngeal cancer, acute lymphoblastic leukemia, all types of acute lymphocytic leukemia, acute myeloid leukemia, adult leukemia, childhood leukemia, chronic lymphocytic leukemia, chronic myeloid leukemia, liver cancer, lobular carcinoma, lung cancer, small cell lung cancer, Hodgkin's lymphoma, non-Hodgkin's lymphoma, malignant glioma, melanoma, meningioma, multiple myeloma, myelodysplastic syndrome, nasopharyngeal cancer, neuroendocrine tumor, oral cancer, osteosarcoma, ovarian cancer, pancreatic cancer, pancreatic neuroendocrine tumors, parathyroid cancer, penile cancer, peritoneal cancer, Peutz-Jeghers syndrome, pituitary gland tumor, polycythemia vera, prostate cancer, renal cell carcinoma, retinoblastoma, salivary gland cancer, sarcoma, Kaposi sarcoma, skin cancer, small intestine cancer, stomach cancer, testicular cancer, thymoma, thyroid cancer, uterine (endometrial) cancer, vaginal cancer, Wilms' tumor.

Cancers that may be mentioned include myelodysplastic syndrome and sub-types, such as acute myeloid leukemia, refractory anemia or refractory anemia with ringed sideroblasts (if accompanied by neutropenia or thrombocytopenia or requiring transfusions), refractory anemia with excess blasts, refractory anemia with excess blasts in transformation, and chronic myeloid (myelomonocytic) leukemia.

Osteporosis drugs that may be mentioned include the bisphosphonates, such as clodronate, ibandronate, pamidronate, zoledronic acid, etidronate, alendronate, risedronate, tiludronate, bondronate and derivatives (e.g. acid derivatives of these compounds).

Other drugs that may be mentioned for use in compositions made by the process of the invention include Immunomodulatory Imide drugs, such as thalidomide and analogues thereof, such as pomalidomide, lenalidomide and apremilast, and pharmaceutically-acceptable salts of any of these, Other drugs that many be mentioned include angiotensin II receptor type 2 agonists, such as Compound 21 (C21; 3-[4-(1H-imidazol-1-ylmethyl)phenyl]-5-(2-methylpropyl)thiophene-2-[(N-butyloxylcarbamate)-sulphonamide] and pharmaceutically-acceptable (e.g. sodium) salts thereof.

Preferred anticancer agents include lenalidomide, which is useful in the treatment of multiple myeloma and anaemia in low to intermediate risk myelodysplastic syndrome and, especially, azacitidine, which is useful in the treatment of certain subtypes of myelodysplastic syndrome. Another specific anticancer drug that may be mentioned is cisplatin, which is a chemotherapeutic agent useful in numerous cancer, including testicular, cervical, ovarian cancer, bladder cancer, lung, esophageal and head and neck cancers, as well as brain tumors, neuroblastoma and mesothelioma.

Other preferred biologically-active agents that may be mentioned include liraglutide, which is useful in the treatment of type 2 diabetes mellitus and prevention of cardiovascular complications associated with diabetes. Particular drugs that may be mentioned in this regards include the lucagon-like peptide-1 receptor agonists, such as exenatide, lixisenatide, albiglutide, dulaglutide, more preferably tirzepatide and semaglutide, and especially liraglutide.

Alternatively, compositions made by a process of the invention may also comprise, instead of (or in addition to) biologically-active agents, diagnostic agents (i.e. agents with no direct therapeutic activity per se, but which may be used in the diagnosis of a condition, such as a contrast agents or contrast media for bioimaging).

Compositions made by a process of the invention may cause an inflammatory response after injection, e.g. subcutaneously. This response may be produced by any component, or combination of components, of such a formulation (including the coatings or carrier system).

Biologically active agents that may in particular be mentioned in this regard include those in which the biologically active agent may, on its own or in the form of a composition made by the process of the invention, produce an inflammatory response when administered to a patient, or may be expected to produce such a response.

In this respect, biologically active agents that may in particular be mentioned for use in compositions made by the process of the invention include, for example, antineoplastic agents, topoisomerase inhibitors, immunomodulators (such as thalidomide, pomalidomide, lenalidomide and apremilast), immunostimulants, immunosuppressants, chemotherapeutics, growth factors, vasodilators and radiopharmaceuticals.

Particular biologically active agents that may be mentioned in this regard include any one or more of the specific anticancer agents listed above and, in particular, actinomycin, azacitidine, azathioprine, bendamustine, bexaroten, bleomycin, bortezomib, bosutinib, busulfan, cabazitaxel, capecitabine, carboplatin, chlorambucil, cladribine, clofarabine, cytarabine, dabrafenib, dacarbazine, dactinomycin, daunorubicin, decitabine, docetaxel, doxifluridine, doxorubicin, epirubicin, epothilone, estramustin, etoposide, everolimus, fludarabine, fluorouracil, guadecitabine, gemcitabine, hydroxyurea, idarubicin, ifosfamide, irinotecan, ixazomib, karfilzomib, lomustin, mechlorethamine, melphalan, mercaptopurine, mesna, methotrexate, mitotan, mitoxantrone, nelarabin, oxaliplatin, paclitaxel, panobinostat, pemetrexed, pixantron, procarbazine, tegafur, temozolomide, teniposide, tioguanine, tiotepa, topotecan, trabektedin, valrubicin, venetoclax, vinblastine, vincristine, vindesine, vinflunine and vinorelbine, as well as pharmaceutically acceptable salts of any of these.

Further biologically active agents that may be mentioned in this respect include certain cytokines, proteins, and vaccines, as well as therapeutic peptides/proteins such as daratumumab, isatuximab and complement C1 esterase inhibitors

Other drugs that may be mentioned in this regard include bendamustine, bleomycin, carboplatin, chlorambucil, cisplatin, cyclophosphamide, cyclosporin, cytarabine, dacarbazine, dactinomycin, daunorubicin, docetaxel, doxorubicin, epirubicin, etoposide, everolimus, fluorouracil, gemcitabine, ifosfamide, irinotecan, mercaptopurine, mesna, methotrexate, midazolam, mitomycin, oxaliplatin, paclitaxel, procarbazine, temsirolimus, thioguanine, vinblastine, vincristine, vinorelbine or pharmaceutically acceptable salts of any of these.

Such a mild inflammatory response may be alleviated by co-administration with an antiinflammatory agent that is suitable for injection.

Appropriate antiinflammatory agents that may be employed in this regard include butylpyrazolidines (such as phenylbutazone, mofebutazone, oxyphenbutazone, clofezone, kebuzone and suxibuzone); acetic acid derivatives and related substances (indomethacin, sulindac, tolmetin, zomepirac, diclofenac, alclofenac, bumadizone, etodolac, lonazolac, fentiazac, acemetacin, difenpiramide, oxametacin, proglumetacin, ketorolac, aceclofenac and bufexamac); oxicams (such as piroxicam, tenoxicam, droxicam, lornoxicam and meloxicam); propionic acid derivatives (such as ibuprofen, naproxen, ketoprofen, fenoprofen, fenbufen, benoxaprofen, suprofen, pirprofen, flurbiprofen, indoprofen, tiaprofenic acid, oxaprozin, ibuproxam, dexibuprofen, flunoxaprofen, alminoprofen, dexketoprofen, vedaprofen, carprofen and tepoxalin); fenamates (such as mefenamic acid, tolfenamic acid, flufenamic acid, meclofenamic acid and flunixin), coxibs (such as celecoxib, rofecoxib, valdecoxib, parecoxib, etoricoxib, lumiracoxib, firocoxib, robenacoxib, mavacoxib and cimicoxib); other non-steroidal antiinflammatory agents (such as nabumetone, niflumic acid, azapropazone, glucosamine, benzydamine, glucosaminoglycan polysulfate, proquazone, orgotein, nimesulide, feprazone, diacerein, morniflumate, tenidap, oxaceprol, chondroitin sulfate, pentosan polysulfate and aminopropionitrile); corticosteroids (such as 11-dehydrocorticosterone, 11-deoxycorticosterone, 11-deoxycortisol, 11-ketoprogesterone, 11β-hydroxypregnenolone, 11β-hydroxyprogesterone, 11β,17α,21-trihydroxypregnenolone, 17α,21-dihydroxypregnenolone, 17α-hydroxypregnenolone, 17α-hydroxyprogesterone, 18-hydroxy-11-deoxycorticosterone, 18-hydroxycorticosterone, 18-hydroxyprogesterone, 21-deoxycortisol, 21-deoxycortisone, 21-hydroxypregnenolone (prebediolone), aldosterone, corticosterone (17-deoxycortisol), cortisol (hydrocortisone), cortisone, pregnenolone, progesterone, flugestone (flurogestone), fluorometholone, medrysone (hydroxymethylprogesterone), prebediolone acetate (21-acetoxypregnenolone), chloroprednisone, cloprednol, difluprednate, fludrocortisone, fluocinolone, fluperolone, fluprednisolone, loteprednol, methylprednisolone, prednicarbate, prednisolone, prednisone, tixocortol, triamcinolone, alclometasone, beclometasone, betamethasone, clobetasol, clobetasone, clocortolone, desoximetasone, dexamethasone, diflorasone, difluocortolone, fluclorolone, flumetasone, fluocortin, fluocortolone, fluprednidene, fluticasone, fluticasone furoate, halometasone, meprednisone, mometasone, mometasone furoate, paramethasone, prednylidene, rimexolone, ulobetasol (halobetasol), amcinonide, budesonide, ciclesonide, deflazacort, desonide, formocortal fluclorolone acetonide (flucloronide), fludroxycortide (flurandrenolone, flurandrenolide), flunisolide, fluocinolone acetonide, fluocinonide, halcinonide and triamcinolone acetonide); quinolines (such as oxycinchophen); gold preparations (such as sodium aurothiomalate, sodium aurothiosulfate, auranofin, aurothioglucose and aurotioprol); penicillamine and similar agents (such as bucillamine); and antihistamines (such as akrivastin, alimemazin, antazolin, astemizol, azatadin, azelastin, bamipin, bilastin, bromdifenhydramin, bromfeniramin, buklizin, cetirizin, cinnarizine, cyklizin, cyproheptadine, deptropine, desloratadin, dexbromfeniramin, dexklorfeniramin, difenylpyralin, dimenhydrinat, dimetinden, doxylamin, ebastin, epinastin, fenindamin, feniramin, fexofenadin, histapyrrodin, hydroxietylprometazin, isotipendyl, karbinoxamin, ketotifen, kifenadin, klemastin, klorcyklizin, klorfenamin, klorfenoxamin, kloropyramin, levocetirizin, loratadin, mebhydrolin, mekitazin, meklozin, mepyramin, metapyrilen, metdilazin, mizolastin, oxatomide, oxomemazine, pimetixen, prometazin, pyrrobutamin, rupatadin, sekifenadin, talastin, tenalidin, terfenadin, tiazinam, tietylperazin, tonzylamin, trimetobenzamid, tripelennamin, triprolidine and tritokvalin). Combinations of any one or more of the above-mentioned antiinflammatory agents may be used.

Preferred antiinflammatory agents include non-steroidal anti-inflammatory drugs, such as diclofenac, ketoprofen, meloxicam, aceclofenac, flurbiprofen, parecoxib, ketorolac, indomethacin or pharmaceutically acceptable salts thereof.

Subjects may receive (or may already be receiving) one or more of the aforementioned co-therapeutic and/or antiinflammatory agents, separate to a composition made by the process of the invention, by which we mean receiving a prescribed dose of one or more of those other therapeutic agents, prior to, in addition to, and/or following, treatment with a composition made by the process of the invention.

When biologically active agents are ‘combined’ with such antiinflammatory agents, the active ingredients may be administered together in the same formulation, or administered separately (simultaneously or sequentially) in different formulations (hereinafter referred to as ‘combination products’).

Such combination products provide for the administration of biologically active agent in conjunction with the antiinflammatory agent, and may thus be presented either as separate formulations, wherein at least one of those formulations is a composition made by the process of the invention, and at least one comprises the antiinflammatory agent in a separate formulation, or may be presented (i.e. formulated) as a combined preparation (i.e. presented as a single formulation including biologically active agent and the antiinflammatory agent).

In this respect an antiinflammatory agent may be co-presented with biologically active agent at an appropriate dose in one or more of the cores that form part of a composition made by the process of the invention as hereinbefore described, or may be formulated using the same or a similar process for coating to that described hereinbefore for the biologically active agent, including a process of the invention, which may allow for the release of the other antiinflammatory agent over the same, or over a different timescale.

Pharmaceutically acceptable salts of biologically active agents include acid addition salts and base addition salts. Such salts may be formed by conventional means, for example by reaction of a free acid or a free base form of a compound of the invention with one or more equivalents of an appropriate acid or base, optionally in a solvent, or in a medium in which the salt is insoluble, followed by removal of said solvent, or said medium, using standard techniques (e.g. in vacuo, by freeze-drying or by filtration). Salts may also be prepared using techniques known to those skilled in the art, such as by exchanging a counter-ion of a compound of the invention in the form of a salt with another counter-ion, for example using a suitable ion exchange resin.

Particular salts that may be mentioned include acid additional salts of, for example, hydrochloric acid, L-lactic acid, acetic acid, phosphoric acid, (+)-L-tartaric acid, citric acid, propionic acid, butyric acid, hexanoic acid, L-aspartic acid, L-glutamic acid, succinic acid, ethylenediaminetetraacetic acid (EDTA), maleic acid, methanesulfonic acid and the like.

Compositions made by the process of the invention may comprise a pharmacologically-effective amount of biologically-active agents. The term ‘pharmacologically-effective amount’ refers to an amount of such active ingredient, which is capable of conferring a desired physiological change (such as a therapeutic effect) on a treated patient, whether administered alone or in combination with another active ingredient. Such a biological or medicinal response, or such an effect, in a patient may be objective (i.e. measurable by some test or marker) or subjective (i.e. the subject gives an indication of, or feels, an effect), and includes at least partial alleviation of the symptoms of the disease or disorder being treated, or curing or preventing said disease or disorder.

Doses of active ingredients that may be administered to a patient should thus be sufficient to affect a therapeutic response over a reasonable and/or relevant timeframe. One skilled in the art will recognize that the selection of the exact dose and composition and the most appropriate delivery regimen will also be influenced by not only the nature of the active ingredient, but also inter alia the pharmacological properties of the formulation, the route of administration, the nature and severity of the condition being treated, and the physical condition and mental acuity of the recipient, as well as the age, condition, body weight, sex and response of the patient to be treated, and the stage/severity of the disease, as well as genetic differences between patients.

Administration of compositions made by the process of the invention may be continuous or intermittent (e.g. by bolus injection). Dosages of active ingredients may also be determined by the timing and frequency of administration.

In any event, the medical practitioner, or other skilled person, will be able to determine routinely the actual dosage of any particular active ingredient, which will be most suitable for an individual patient.

Non-biologically active adjuvants, diluents and carriers that may be employed in cores to be coated in accordance with the invention may include pharmaceutically-acceptable substances that are soluble in water, such as carbohydrates, e.g. sugars, such as lactose and/or trehalose, and sugar alcohols, such as mannitol, sorbitol and xylitol; or pharmaceutically-acceptable inorganic salts, such as sodium chloride. Preferred carrier/excipient materials include sugars and sugar alcohols. Such carrier/excipient materials are particularly useful when the biologically active agent is a complex macromolecule, such as a peptide, a protein or portions of genetic material or the like, for example as described generally and/or the specific peptides/proteins described hereinbefore including vaccines. Embedding complex macromolecules in excipients in this way will often result in larger cores for coating, and therefore larger coated particles.

In addition to comprising one or more biologically active agent, the cores may comprise one or more non-biologically active adjuvants, diluents and carriers, including emollients, and/or other excipients with a functional property, such as a buffering agent and/or a pH modifying agent (e.g. citric acid).

When injected, formulations produced by the process of the invention provide a depot formulation, from which biologically active agent is released over a prolonged period of time. That period of time may be at least about 3 days, such as about 5, or about 7, days, and up to a period of about a year, such as about 3 weeks (e.g. about 2 weeks or about 4 weeks), or about 12 weeks (e.g. about 10 weeks or about 14 weeks).

The solid cores are provided in the form of nanoparticles or, more preferably, microparticles. Preferred weight-, number-, or volume-based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

As used herein, the term ‘weight based mean diameter’ will be understood by the skilled person to include that the average particle size is characterised and defined from a particle size distribution by weight, i.e. a distribution where the existing fraction (relative amount) in each size class is defined as the weight fraction, as obtained by e.g. sieving (e.g. wet sieving). As used herein, the term ‘number based mean diameter’ will be understood by the skilled person to include that the average particle size is characterised and defined from a particle size distribution by number, i.e. a distribution where the existing fraction (relative amount) in each size class is defined as the number fraction, as measured by e.g. microscopy. As used herein, the term ‘volume based mean diameter’ will be understood by the skilled person to include that the average particle size is characterised and defined from a particle size distribution by volume, i.e. a distribution where the existing fraction (relative amount) in each size class is defined as the volume fraction, as measured by e.g. laser diffraction. The person skilled in the art will also understand there are other suitable ways of expressing mean diameters, such as area based mean diameters, and that these other expressions of mean diameter are interchangeable with those used herein. Other instruments that are well known in the field may be employed to measure particle size, such as equipment sold by e.g. Malvern Instruments, Ltd (Worcestershire, UK) and Shimadzu (Kyoto, Japan).

The solid cores may have a preferred weight-, number-, or volume-based mean diameters are between about 50 nm (e.g. about 100 nm, such as about 250 nm) and about 30 μm, for example between about 500 nm and about 100 μm, more particularly between about 1 μm and about 50 μm, such as about 25 μm, e.g. about 20 μm.

In the process of the invention, at least 200 mg of the solid cores may be loaded into the stationary gas phase deposition reactor chamber for the gas phase deposition technique to be applied, optionally at least 1 g or at least 10 g.

Particles may be spherical, that is they possess an aspect ratio smaller than about 20, more preferably less than about 10, such as less than about 4, and especially less than about 2, and/or may possess a variation in radii (measured from the centre of gravity to the particle surface) In at least about 90% of the particles that is no more than about 50% of the average value, such as no more than about 30% of that value, for example no more than about 20% of that value.

Nevertheless, the coating of particles on any shape is also possible in accordance with the invention. For example, irregular shaped (e.g. ‘raisin’-shaped), needle-shaped, flake-shaped or cuboid-shaped particles may be coated. For a non-spherical particle, the size may be indicated as the size of a corresponding spherical particle of e.g. the same weight, volume or surface area. Hollow particles, as well as particles having pores, crevices etc., such as fibrous or ‘tangled’ particles may also be coated in accordance with the Invention.

Particles may be obtained in a form in which they are suitable to be coated or be obtained in that form, for example by particle size reduction processes (e.g. crushing, cutting, milling or grinding) to a specified weight based mean diameter (as hereinbefore defined), for example by wet grinding, dry grinding, air jet milling (including cryogenic micronization), ball milling, such as planetary ball milling, as well as making use of end-runner mills, roller mills, vibration mills, hammer mills, roller mill, fluid energy mills, pin mills, etc. Alternatively, particles may be prepared directly to a suitable size and shape, for example by spray-drying, freeze-drying, spray-freeze-drying, vacuum-drying, precipitation, including the use of supercritical fluids or other top-down methods (i.e. reducing the size of large particles, by e.g. grinding, etc.), or bottom-up methods (i.e. increasing the size of small particles, by e.g. sol-gel techniques, crystallization, etc.). Nanoparticles may alternatively be made by well-known techniques, such as gas condensation, attrition, chemical precipitation, ion implantation, pyrolysis, hydrothermal synthesis, etc.

It may be necessary (depending upon how the particles that comprise the cores are initially provided) to wash and/or clean them to remove impurities that may derive from their production, and then dry them. Drying may be carried out by way of numerous techniques known to those skilled in the art, including evaporation, spray-drying, vacuum drying, freeze drying, fluidized bed drying, microwave drying, IR radiation, drum drying, etc. If dried, cores may then be deagglomerated by grinding, screening, milling and/or dry sonication. Alternatively, cores may be treated to remove any volatile materials that may be absorbed onto its surface, e.g. by exposing the particle to vacuum and/or elevated temperature.

Surfaces of cores may be chemically activated prior to applying the first layer of coating material, e.g. by treatment with hydrogen peroxide, ozone, free radical-containing reactants or by applying a plasma treatment, in order to create free oxygen radicals at the surface of the core. This in turn may produce favourable adsorption/nucleation sites on the cores for reactants (which reactants may hereinafter be referred to interchangeably as ‘precursors’) used to deposit coating material by a gas phase deposition technique.

More than one layer of coating material is applied to the core sequentially. Preferred gas phase deposition techniques include ALD or related technologies, such as atomic layer epitaxy (ALE), molecular layer deposition (MLD; a similar technique to ALD with the difference that molecules (commonly organic molecules) are deposited in each pulse instead of atoms), molecular layer epitaxy (MLE), chemical vapor deposition (CVD), atomic layer CVD, molecular layer CVD, physical vapor deposition (PVD), sputtering PVD, reactive sputtering PVD, evaporation PVD and binary reaction sequence chemistry. ALD is the preferred method of coating according to the invention.

When ALD is employed, the coating materials may be prepared by feeding a precursor, or reactant, into an ALD reactor chamber (in a so called ‘precursor pulse’) to form the adsorbed atomic or molecular monolayer at the surface of the particle. A subsequent purging pulse is followed by a second precursor pulse into the reactor which reacts with the first precursor, resulting in the formation of a monolayer of a compound on the substrate surface. Another purging pulse is followed by a further pulse of the first precursor, and thus the start of a new cycle of the same events, which is an ALD cycle.

In most instances, the first of the consecutive reactions will involve some functional group or free electron pairs or radicals at the surface to be coated, such as a hydroxy group (—OH) or a primary or secondary amino group (—NH2 or —NHR where R e.g. is an aliphatic group, such as an alkyl group). The individual reactions are advantageously carried out separately and under conditions such that all excess reagents and reaction products are essentially removed before conducting the subsequent reaction.

Two or more separate layers or coating material (also referred to herein as ‘coatings’ or ‘shells’, all of which terms are used herein interchangeably) are applied (that is ‘separately applied’) to the solid cores comprising biologically active agent. Such ‘separate application’ of ‘separate layers, coatings or shells’ means that the solid cores are coated with a first layer of coating material, which layer is formed by more than one (e.g. a plurality or a set of) cycles as described herein, each cycle producing a monolayer of coating material, and then that resultant coated core may be subjected to some form of sieving step, such as a vibrational sieving technique, step or process as described herein.

In other words, ‘gas-phase deposition (e.g. ALD) cycles’ can be repeated several times to provide a ‘gas-phase deposition (e.g. ALD) set’ of cycles, which may consist of e.g. 10, 25 or 100 cycles. However, after this set of cycles, the coated core may be subjected to some form of sieving step, such as a vibrational sieving technique, step or process as described herein, which is then followed by a further set of cycles.

This process may be repeated as many times as is desired and, in this respect, the number of discrete layers of coating material(s) as defined herein corresponds to the number of these intermittent sieving steps. Optionally, at least one of those sieving steps comprises a vibrational sieving step. In some examples, at least the final sieving step comprises the vibrational sieving step being conducted prior to the application of a final layer (set of cycles) of coating material. In further examples, more than one (including each) of the sieving steps comprise vibrational sieving techniques, steps or processes as described herein.

The vibrational sieving technique may comprise a vibration motor coupled to a sieve, and provides a means of vibrationally forcing the solid product mass formed by coating said cores through a sieve that may be located internally or (preferably) externally to (i.e. outside of) the reactor, and is configured to deagglomerate any particle aggregates upon said vibrational forcing of the coated cores, prior to being subjected to a second and/or a further layer of coating material. This process may be repeated as many times as is required and/or appropriate prior to the application of a final layer of coating material.

Vibrational forcing means may comprise a vibration motor which is coupled to a sieve. The vibration motor is configured to vibrate and/or gyrate when an electrical power is supplied to it. For example, the vibration motor may be a piezoelectric vibration motor comprising a piezoelectric material which changes shape when an electric field is applied, as a consequence of the converse piezoelectric effect. The changes in shape of the piezoelectric material cause acoustic or ultrasonic vibrations of the piezoelectric vibration motor.

The vibration motor may alternatively be an eccentric rotating mass (ERM) vibration motor comprising a mass which is rotated when electrical power is supplied to the motor. The mass is eccentric from the axis of rotation, causing the motor to be unbalanced and vibrate and/or gyrate due to the rotation of the mass. Further, the ERM vibration motor may comprise a plurality of masses positioned at different locations relative to the motor. For example, the ERM vibration motor may comprise a top mass and a bottom mass each positioned at opposite ends of the motor. By varying each mass and its angle relative to the other mass, the vibrations and/or gyrations of the ERM vibration motor can be varied.

The vibration motor may be coupled to the sieve in a manner in which vibrations and/or gyrations of the motor when electrical power is supplied to it are transferred to the sieve.

The sieve and the vibration motor may be suspended from a mount (such as a frame positionable on a floor, for example) via a suspension means such that the sieve and motor are free to vibrate relative to the mount without the vibrations being substantially transferred to or dampened by the mount. This allows the vibration motor and sieve to vibrate and/or gyrate without impediment and also reduces noise generated during the vibrational sieving process. The suspension means may comprise one or more springs or bellows (i.e. air cushion or equivalent cushioning means) that couple the sieve and/or motor to the mount. Manufacturers of vibratory sieves or sifters suitable for carrying out such a process include for instance Russell Finex, SWECO, Filtra Vibracion, VibraScreener, Gough Engineering and Farley Greene.

Preferably, the vibrational sieving technique further comprises controlling a vibration probe coupled to the sieve. The vibration probe may be controlled to cause the sieve to vibrate at a separate frequency to the frequency of vibrations caused by the vibration motor. Preferably the vibration probe causes the sieve to vibrate at a higher frequency than the vibrations caused by the vibration motor and, more preferably, the frequency is within the ultrasonic range.

Providing additional vibrations to the sieve by means of the vibration probe reduces the occurrence of clogging in the sieve, reduces the likelihood of the sieve being overloaded and decreases the amount of time needed to clean the mesh of the sieve.

When each and every sieving step does not comprise such a vibrational sieving technique, step, sieving steps can nevertheless be conducted by one or more other means of forcing the coated mass through a sieve in a manual, mechanical and/or automated way. Mechanical forces may take the form of tapping, oscillation, application of a pressure gradient (e.g. a jet), horizontal rotation, mechanised periodical displacement of a sieve, centrifugal forces, sieving or combinations thereof, such as oscillating and tapping, rotating and tapping, etc.

Such alternative forcing means are preferably mechanical and may also be vibrational, in which an appropriate alternative means of applying a vibrational force (i.e. one that does not comprise a vibration motor coupled to a sieve) forces the coated mass of powder through a mesh or sieve. Alternative mechanical means of generating oscillations about an equilibrium point may comprise acoustic waves (including sonic and ultrasonic waves), or may be mechanical (e.g. tapping), or other ways, including combinations thereof, such as ultrasonic and sonic, sonic and tapping, ultrasonic and tapping, etc.

In such cases, we prefer that at least one of these alternative mechanical sieving steps is carried out by way of a sonic sifter, as described hereinafter. Manufacturers of suitable sonic sifters include Advantech Manufacturing, Endecott and Tsutsui.

Preferably, the vibrational sieving technique comprises sieving coated particles with a throughput of at least 1 g/minute. More preferably, the vibrational sieving technique comprises sieving coated particles with a throughput of 4 g/minute or more.

The throughput depends on the area of the sieve mesh, mesh-size of the sieve, the particle size, the stickiness of the particles, static nature of the particle. By combining some of these features a much higher throughput is possible. Accordingly, the vibrational sieving technique may more preferably comprise sieving coated particles with a throughput of up to 1 kg/minute or even higher.

Any one of the above-stated throughputs represents a significant improvement over the use of known mechanical sieving, or sifting, techniques. For example, we found that sonic sifting involved sifting in periods of 15 minutes with a 15-minute cooling time in-between, which is necessary for preserving the apparatus. To sift 20 g of coated particles required 9 sets of 15 minutes of active sifting time, i.e. a total time (including the cooling) of 255 minutes. By comparison, by using the vibrational sieving technique essential to the process of the invention, 20 g of coated particles may be sieved continuously in, at most, 20 minutes, or more preferably in just 5 minutes, or less.

The sieve mesh size may be determined so that the ratio of the size of the sieved or sonic sifted particles to the sieve mesh size is about 1:>1, preferably about 1:2, and optionally about 1:4. The size mesh size may range from about 20 μm to about 100 μm, preferably from about 20 μm to about 60 μm.

Appropriate sieve meshes may include perforated plates, microplates, grid, diamond, threads, polymers or wires (woven wire sieves) but are preferably formed from metals, such as stainless steel.

Surprisingly, using a stainless steel mesh within such a vibrational sieving technique is as gentle to the particle coatings as using a softer polymer sieve as part of a mechanical sieving technique such as sonic sifting.

Also, a known problem with sieving powders is the potentially dangerous generation of static electricity. A steel mesh has the advantage of removing static electricity from the powder while that is not the case with a polymeric mesh, which has to be used in a sonic sifter.

Further, the mesh size of known sonic sifters is limited to about 100 μm since the soundwaves travel through the mesh rather than vibrating it. That limitation does not exist using for vibrational sieving techniques as there is no reliance on soundwaves to generate vibrations in the sieve. Therefore, the vibrational sieving technique as described herein allows larger particles to be sieved than if alternative mechanical sieving techniques were used.

If a (e.g. vibrational) sieve is located externally to (i.e. outside of) the reactor, step (2) of the process of the invention comprises discharging the coated particles from the gas phase deposition reactor prior to subjecting the coated particles to agitation, and step (3) comprises reintroducing the deagglomerated, coated particles from step (2) into the gas phase deposition reactor prior to applying a further layer of at least one coating material to the reintroduced particles.

Alternatively, coated cores may also be subjected to the aforementioned vibrational sieving step(s) internally, without being removed from said apparatus by way of a continuous process. Such a process will involve a means of vibrationally forcing the solid product mass formed by coating said cores through a sieve that is located within the reactor, and is configured to deagglomerate any particle aggregates upon said vibrational sieving of the coated cores by means of a forcing means applied within said reactor, prior to being subjected to a second and/or a further coating. This process is continued for as many times as is required and/or appropriate prior to the application of the final coating as described herein.

Having the sieve located within the reactor vessel means that the coating can be applied by way of a continuous process which does not require the particles to be removed from the reactor. Thus, no manual handling of the particles is required, and no external machinery is required to deagglomerate the aggregated particles. This not only considerably reduces the time of the coating process being carried out, but is also more convenient and reduces the risk of harmful (e.g. poisonous) materials being handled by personnel. It also enhances the reproducibility of the process by limiting the manual labour and reduces the risk of contamination.

We have found that applying separate layers of coating materials following external deagglomeration gives rise to visible and discernible interfaces that may be observed by analysing coated particles according to the invention, and are observed by e.g. TEM as regions of higher electron permeability. In this respect, the thickness of the layers between interfaces correspond directly to the number of cycles in each series that are carried out within the ALD reactor, and between individual external agitation steps.

Because, in an ALD coating process, coating takes place at the atomic level, such clear, physical interfaces are typically more difficult to observe.

Without being limited by theory, it is believed that removing coated particles from the vacuum conditions of the ALD reactor and exposing a newly-coated surface to the atmosphere results in structural rearrangements due to relaxation and reconstruction of the outermost atomic layers. Such a process is believed to involve rearrangement of surface (and near surface) atoms, driven by a thermodynamic tendency to reduce surface free energy.

Furthermore, surface adsorption of species, e.g. hydrocarbons that are always present in the air, may contribute to this phenomenon, as can surface modifications, due to reaction of coatings formed with hydrocarbons, as well as atmospheric oxygen and the like. Accordingly, if such interfaces are analysed chemically, they may contain traces of contaminants or the core material, such as an API forming part of the core, that do not originate from the coating process, such as ALD.

Whether carried out Inside or outside of the reactor, particle aggregates are thus broken up by a vibrational forcing means that forces them through a sieve, thus separating the aggregates into individual particles or aggregates of a desired and predetermined size (and thereby achieving deagglomeration). In the latter regard, in some cases the individual primary particle size is so small (i.e. <1 μm) that achieving ‘full’ deagglomeration (i.e. where aggregates are broken down into individual particles) is not possible. Instead, deagglomeration is achieved by breaking down larger aggregates into smaller aggregates of secondary particles of a desired size, as dictated by the size of the sieve mesh. The smaller aggregates are then coated by the gas phase technique to form fully coated ‘particles’ in the form of small aggregate particles. In this way, the term ‘particles’, when referring the particles that have been deagglomerated and coated in the context of the invention, refers to both individual (primary) particles and aggregate (secondary) particles of a desired size.

In any event, the desired particle size (whether that be of individual particles or aggregates of a desired size) is maintained and, moreover, continued application of the gas phase coating mechanism to the particles after such deagglomeration via the vibrational sieving means that a complete coating is formed on the particle, thus forming fully-coated particles (individual or aggregates of a desired size).

Whether carried out inside or outside of the reactor, the process of the invention may be carried out in a manner that involves carrying out the above-described repeated coating and deagglomeration/agitation process at least 1, preferably 2, more preferably 3, such as 4, including 5, more particularly 6, e.g. 7 times, and no more than about 100 times, for example no more than about 50 times, such as no more than about 40 times, including no more than about 30 times, such as between 2 and 20 times, e.g. between 3 and 15 times, such as 10 times, e.g. 9 or 8 times, more preferably 6 or 7 times, and particularly 4 or 5 times.

Whether carried out inside or outside of the reactor, it is preferred that at least one sieving step is carried out and further that that step preferably comprises a vibrational sieving step as described above. It is further preferred that at least the final sieving step comprises a vibrational sieving step being conducted prior to the application of a final layer (set of cycles) of coating material. However, it is further preferred that more than one (including each) of the sieving steps comprise vibrational sieving techniques, steps or processes as described herein.

The preferable repetition of these steps makes the improved throughput of any vibrational sieving technique all the more beneficial.

The total thickness of the coating (meaning all the separate layers/coatings/shells) will on average be in the region of between about 0.5 nm and about 2 μm.

The minimum thickness of each individual layer/coating/shell will on average be in the region of about 0.1 nm (for example about 0.5 nm, or about 0.75 nm, such as about 1 nm).

The maximum thickness of each individual layer/coating/shell will depend on the size of the core (to begin with), and thereafter the size of the core with the coatings that have previously been applied, and may be on average about 1 hundredth of the mean diameter (i.e. the weight-, number-, or volume-, based mean diameter) of that core, or core with previously-applied coatings.

Preferably, for particles with a mean diameter that is between about 100 nm and about 1 μm, the total coating thickness should be on average between about 1 nm and about 5 nm; for particles with a mean diameter that is between about 1 μm and about 20 μm, the coating thickness should be on average between about 1 nm and about 10 nm; for particles with a mean diameter that is between about 20 μm and about 700 μm, the coating thickness should be on average between about 1 nm and about 100 nm.

We have found that applying coatings/shells followed by conducting one or more deagglomeration step such as sonication gives rise to abrasions, pinholes, breaks, gaps, cracks and/or voids (hereinafter ‘cracks’) in the layers/coatings, due to coated particles essentially being more tightly ‘bonded’ or ‘glued’ together directly after the application of a thicker coating. This may expose a core comprising biologically-active ingredient to the elements once deagglomeration takes place.

As described in international patent application PCT/GB2020/053129, we have surprisingly found that conducting a mechanical sifting process (as opposed to sonication as described in international patent application WO 2014/187995, manually forcing the particles through a sieve by hand, or by the mechanical sifting process mentioned in international patent application PCT/GB2020/053129) gives rise to significantly less pinholes, gaps or cracks in the coating material. This, in turn, gives rise to particles that are not only completely covered by that layer/coating, but are also covered in a manner that enables the particles to be deagglomerated readily (e.g. using a non-aggressive technique, such as vortexing) in a manner that does not destroy the layers of coating material that have been formed, prior to, and/or during, pharmaceutical formulation.

We have, very surprisingly, also found that the above-mentioned low frequency of pinholes, gaps or cracks in the coating material can be maintained when using a vibrational sieving technique. This was surprising given that the technique described herein employs a stainless steel sieve (rather than a softer, polymer sieve used in the mechanical sifting process mentioned in international patent application PCT/GB2020/053129), as may be preferred for such vibrational sieving techniques. Previous attempts to manually force particles through metallic sieves gave rise to the significant formation of pinholes, gaps or cracks in the coating material.

For example, if it is intended to provide a sample in suspension prior to administration to a patient, it is necessary to provide deagglomerated primary particles without pinholes or cracks in the coatings. Such cracks will result in an undesirable initial peak (burst) in plasma concentration of active ingredient directly after administration.

Processes described herein result in the deagglomerated coated particles with the essential absence of said cracks through which active ingredient can be released in an uncontrolled way. By ‘essentially free of said cracks’ in the coating(s), we mean that less than about 1% of the surfaces of the coated particles comprise abrasions, pinholes, breaks, gaps, cracks and/or voids through which active ingredient is potentially exposed (to, for example, the elements).

In this respect, the (e.g. inorganic, such as mixed oxide) coating typically completely surrounds, encloses and/or encapsulates said solid cores. In this way, the risk of an initial drug concentration burst due to the drug coming into direct contact with solvents in which the relevant active ingredient is soluble is minimized. This may include not only bodily fluids, but also any medium in which such coated particles may be suspended prior to injection.

Thus in one embodiment of the invention, there are provided particles as hereinbefore disclosed, wherein said coating surrounding, enclosing and/or encapsulating said core covers at least about 50%, such as at least about 65%, including at least about 75%, such as at least about 80%, more particularly at least about 90%, such as at least about 91%, such as at least about 92%, such as at least about 93%, such as at least about 94%, such as at least about 95%, such as at least about 96%, such as at least about 97%, such as at least about 98%, such as at least about 99%, such as approximately, or about, 100%, of the surface of the solid core, such that the coating essentially completely surrounds, encloses and/or encapsulates said core.

As used herein, the term ‘essentially completely coating completely surrounds, encloses and/or encapsulates said core’ means a covering of at least about 98%, or at least about 99%, of the surface of the solid core.

Although some minor cracks may appear in the said coating without effecting the essential function thereof in terms of controlling release, in a further embodiment, there are provided particles as hereinbefore disclosed, wherein at least about 90% of the particles do not exhibit cracks in the coating surrounding, enclosing and/or encapsulating said core. In one embodiment at least about 91%, such as at least about 92%, such as at least about 93%, such as at least about 94%, such as at least about 95%, such as at least about 96%, such as at least about 97%, such as at least about 98%, such as at least about 99%, such as approximately 100% of the particles do not exhibit said cracks.

The layers of coating material may, taken together, be of an essentially uniform thickness over the surface area of the particles. By ‘essentially uniform’ thickness, we mean that the degree of variation in the thickness of the coating of at least about 10%, such as about 25%, e.g. about 50%, of the coated particles that are present in a composition made by a process of the Invention, as measured by TEM, Is no more than about ±20%, including ±50% of the average thickness.

Coating materials that may be applied to cores may be pharmaceutically-acceptable, in that they should be essentially non-toxic.

Coating materials may comprise organic or polymeric materials, such as a polyamide, a polyimide, a polyurea, a polyurethane, a polythiourea, a polyester or a polyimine. Coating materials may also comprise hybrid materials (as between organic and inorganic materials), including materials that are a combination between a metal, or another element, and an alcohol, a carboxylic acid, an amine or a nitrile. However, we prefer that coating materials comprise inorganic materials.

Inorganic coating materials may comprise one or more metals or metalloids, or may comprise one or more metal-containing, or metalloid-containing, compounds, such as metal, or metalloid, oxides, nitrides, sulphides, selenides, carbonates, and/or other ternary compounds, etc. Metal, and metalloid, hydroxides and, especially, oxides are preferred, especially metal oxides.

Metals that may be mentioned include alkali metals, alkaline earth metals, noble metals, transition metals, post-transition metals, lanthanides, etc. Metal and metalloids that may be mentioned include aluminium, titanium, magnesium, iron, gallium, zinc, zirconium, niobium, hafnium, tantalum, lanthanum, and/or silicon; more preferably aluminium, titanium, magnesium, iron, gallium, zinc, zirconium, and/or silicon; especially aluminium, silicon, titanium and/or zinc.

The metal or metalloid-containing precursor may be applied as either the first or the second reactant gas during the gas phase deposition technique. In some processes, it may be advantageous to start with the metal or metalloid-containing precursor whereas in others it may be advantageous to start with an oxygen precursor, such as water. This may be dependent on the moieties on the surface of the compound to be coated, for example.

In embodiments of the invention, each repetition of applying the gas phase deposition technique may be carried out using the same, or different, first and second reactant gases to the previous iteration of step (b). For example, the metal or metalloid-containing precursor used in one iteration may be different to the metal or metalloid-containing precursor used in the subsequent iteration. Further, in embodiments of the invention involving deagglomeration of particles between repetitions of the gas phase deposition technique, the metal or metalloid-containing precursor may alternate from being the first reactant gas to being the second reactant gas or vice versa.

As mentioned above, as the compositions made by the process of the invention may comprise two or more discrete layers of inorganic coating materials, the nature and chemical composition(s) of those layers may differ from layer to layer.

Individual layers may also comprise a mixture of two or more inorganic materials, such as metal oxides or metalloid oxides, and/or may comprise multiple layers or composites of different inorganic or organic materials, to modify the properties of the layer.

Coating materials that may be mentioned include those comprising aluminium oxide (Al2O3), titanium dioxide (TiO2), Iron oxides (FexOy, e.g. FeO and/or Fe2O3 and/or Fe3O4), gallium oxide (Ga2O3), magnesium oxide (MgO), zinc oxide (ZnO), niobium oxide (Nb2O5), hafnium oxide (HfO2), tantalum oxide (Ta2O5), lanthanum oxide (La2O3), zirconium dioxide (ZrO2) and/or silicon dioxide (SiO2), Preferred coating materials include aluminium oxide, titanium dioxide, iron oxides, gallium oxide, magnesium oxide, zinc oxide, zirconium dioxide and silicon dioxide. More preferred coating materials include iron oxide, titanium dioxide, zinc sulphide, more preferably zinc oxide, silicon dioxide and/or aluminium oxide.

Layers of coating materials (on an individual or a collective basis) in compositions made by the process of the invention may consist essentially (e.g. is greater than about 80%, such as greater than about, 90%, e.g. about 95%, such as about 98%) of iron oxides, titanium dioxide, or more preferably zinc oxide, silicon oxide and/or aluminium oxide.

The process of the invention is particularly useful when the coating material(s) that is/are applied to the cores comprise zinc oxide, silicon dioxide and/or aluminium oxide.

It is further preferred that the inorganic coating material comprising mixture of:
(i) zinc oxide (ZnO); and
(ii) one or more other metal and/or metalloid oxides,
   wherein the atomic ratio ((i):(ii)) is between at least about 1:6 and up to and including about 6:1.

The coating of comprising a mixture of zinc oxide and one or more other metal and/or metalloid oxides is referred to hereinafter as a ‘mixed oxide’ coating or coating material(s).

The biologically active agent-containing cores may thus be coated with a coating material that comprises a mixture of zinc oxide, and one or more other metal and/or metalloid oxides, at an atomic ratio of zinc oxide to the other oxide(s), or vice versa, that is at least about 1:1 (e.g. at least about 1.5:1, such as at least about 2:1), including at least about 2.25:1, such as at least about 2.5:1 (e.g. at least about 3.25:1 or least about 2.75:1 (including 3:1)), and is up to (i.e, no more than) and including about 6:1, including up to about 5.5:1, or up to about 5:1, such as up to about 4.5:1, including up to about 4:1 (e.g. up to about 3.75:1).

In order to make a mixed oxide coating with an atomic ratio of between about 1:1 and up to and including about 6:1 of zinc oxide relative to the one or more other metal and/or metalloid oxides, the skilled person will appreciate that for every one ALD cycle (i.e. monolayer) of the other oxide(s), between about 1 and about 6 ALD cycles of zinc oxide must also be deposited. For example, for a 3:1 atomic ratio (zinc:other oxide) mixed oxide coating to be formed, 3 zinc-containing precursor pulses may each be followed by second precursor pulses, forming 3 monolayers of zinc oxide, which will then be followed by 1 pulse of the other metal and/or metalloid-containing precursor followed by second precursor pulse, forming 1 monolayer of oxide of the other metal and/or metalloid. Alternatively, 6 monolayers of zinc oxide may be followed by 2 monolayers of the other oxide, or any other combination so as to provide an overall atomic ratio of about 3:1. In this respect, the order of pulses to produce the relevant oxides is not critical, provided that the resultant atomic ratio is in the relevant range in the end.

Other coating materials, such as pharmaceutically-acceptable and essentially non-toxic coating materials may also be applied in addition, either between separate coatings as described herein (e.g. in-between separate deagglomeration steps) and/or whilst a coating is being applied. Such materials may comprise multiple layers or composites of said mixed oxide and one or more different inorganic or organic materials, to modify the properties of the layer(s).

In ALD, layers of coating materials may be applied at process temperatures from about 20° C. to about 800° C., or from about 40° C. to about 200° C., e.g. from about 40° C. to about 150°, such as from about 50° C. to about 100° C. The optimal process temperature depends on the reactivity of the precursors and/or the substances (Including biologically-active agents) that are employed in the core and/or melting point of the core substance(s). When the cores to be coated comprise a biologically-active ingredient, it is preferred that a lower temperature, such as from about 20° C. to about 100° C. is employed. In particular, in one embodiment of the method a temperature from about 20° C. to about 80° C. is employed, such as from about 30° C. to about 70° C., such as from about 40° C. to about 60° C., such as about 50° C.

We have found that, when coatings comprising zinc oxide are applied using ALD at a lower temperature, such as from about 50° C. to about 100° C. (unlike other coating materials, such as aluminium oxide and titanium oxide, which form amorphous layers) the coating materials are largely crystalline in their nature.

Without being limited by theory, because zinc oxide is crystalline, if only zinc oxide is employed as coating material, we are of the understanding that interfaces may be formed between adjacent crystals of zinc oxide that are deposited by ALD, through which a carrier system, medium or solvent in which zinc oxide is partially soluble (e.g. an aqueous solvent system) can ingress following suspension therein. It is believed that this may give rise to dissolution that is too fast for the depot-forming composition that it is intended to make.

We have now found that these problems may be alleviated by making a mixed oxide coating as described herein. In particular, we have now found that these problems may be alleviated by making a mixture of two or more metal and/or metalloid oxides (mixed oxide) coating as described herein. In particular, by forming a mixed oxide coating as described herein, that may be predominantly, but not entirely, comprised of zinc oxide, we have been able to coat active ingredients with coatings that appear to be essentially amorphous, or a composite between crystalline and amorphous material, and/or in which ingress of injection vehicles such as water may be reduced. In this respect, it appears to us that the presence of the aforementioned perceived interfaces may be reduced, or avoided altogether, by employing a mixed oxide according to this aspect of the invention, in either a heterogeneous manner (in which the other oxide is ‘filling in’ gaps formed by the interfaces), or in a homogeneous manner (In which a true composite of mixed oxide materials is formed during deposition, in a manner where the interfaces are potentially avoided in the first place).

There is thus provided a method of preparing of plurality of coated particles in accordance with the invention, wherein the coated particles are made by applying precursors of at least two metal and/or metalloid oxides forming a mixed oxide on the solid cores, and/or previously-coated solid cores, by a gas phase deposition technique. Precursors for forming a metal oxide or a metalloid oxide often include an oxygen precursor, such as water, oxygen, ozone and/or hydrogen peroxide; and a metal and/or metalloid compound, typically an organometal compound or an organometalloid compound.

Non-limiting examples of precursors are as follows: Precursors for zinc oxide may be water and diC1-C5alkylzinc, such as diethylzinc. Precursors for aluminium oxide may be water and triC1-C5alkylaluminium, such as trimethylaluminium. Precursors for silicon oxide (silica) may be water as the oxygen precursor and silanes, alkylsilanes, aminosilanes, and orthosilicic acid tetraethyl ester. Precursors for iron oxide includes oxygen, ozone and water as the oxygen precursor; and di C1-C5alkyl-iron, dicyclopropyl-iron, and FeCl3. It will be appreciated that the person skilled in the art is aware of what precursors are suitable for the purpose as disclosed herein.

Although the plurality of coated particles produced according to the process of the invention are essentially free of the aforementioned cracks in the applied coatings, through which active ingredient is potentially exposed (to, for example, the elements), two further, optional steps may be applied to the plurality of coated particles prior to subjecting it to further pharmaceutical formulation processing.

The first optional step may comprise, subsequent to the final deagglomeration step as hereinbefore described, application of a final overcoating layer, the thickness of which outer ‘overcoating’ layer/coating, or ‘sealing shell’ (which terms are used herein interchangeably), must be thinner than the previously-applied separate layers/coatings/shells (or ‘subshells’).

The thickness may therefore be on average no more than a factor of about 0.7 (e.g. about 0.6) of the thickness of the widest previously-applied subshell. Alternatively, the thickness may be on average no more than a factor of about 0.7 (e.g. about 0.6) of the thickness of the last subshell that is applied, and/or may be on average no more than a factor of about 0.7 (e.g. about 0.6) of the average thickness of all of the previously-applied subshells. The thickness may be on average in the region of about 0.3 nm to about 10 nm, for particles up to about 20 μm. For larger particles, the thickness may be on average no more than about 1/1000 of the coated particles' weight-, number-, or volume-based mean diameter.

The role of such as sealing shell is to provide a ‘sealing’ overcoating layer on the particles, covering over those cracks, so giving rise to particles that are not only completely covered by that sealing shell, but also covered in a manner that enables the particles to be deagglomerated readily (e.g. using a non-aggressive technique, such as vortexing) in a manner that does not destroy the subshells that have been formed underneath, prior to, and/or during, pharmaceutical formulation.

For the reasons described herein, it is preferred that the sealing shell does not comprise zinc oxide. The sealing shell may on the other hand comprise silicon dioxide or, more preferably, aluminium oxide.

The second optional step may comprise ensuring that the few remaining particles with broken and/or cracked shells/coatings are subjected to a treatment in which all particles are suspended in a solvent in which the active ingredient is soluble (e.g. with a solubility of at least about 1 mg/mL), but the least soluble material in the coating is Insoluble (e.g. with a solubility of no more than about 0.1 μg/mL), followed by separating solid matter particles from solvent by, for example, centrifugation, sedimentation, flocculation and/or filtration, resulting in mainly intact particles being left.

The above-mentioned optional step provides a means of potentially reducing further the likelihood of a (possibly) undesirable initial peak (burst) in plasma concentration of active ingredient, as discussed hereinbefore.

At the end of the process, coated particles may be dried using one or more of the techniques that are described hereinbefore for drying cores. Drying may take place in the absence, or in the presence, of one or more pharmaceutically acceptable excipients (e.g. a sugar or a sugar alcohol).

Alternatively, at the end of the process, separated particles may be resuspended in a solvent (e.g. water, with or without the presence of one or more pharmaceutically acceptable excipients as defined herein), for subsequent storage and/or administration to patients.

Prior to applying the first layer of coating material or between successive coatings, cores and/or partially coated particles may be subjected to one or more alternative and/or preparatory surface treatments. In this respect, one or more intermediary layers comprising different materials (i.e. other than the inorganic material(s)) may be applied to the relevant surface, e.g. to protect the cores or partially-coated particles from unwanted reactions with precursors during the coating step(s)/deposition treatment, to enhance coating efficiency, or to reduce agglomeration.

An intermediary layer may, for example, comprise one or more surfactants, with a view to reducing agglomeration of particles to be coated and to provide a hydrophilic surface suitable for subsequent coatings. Suitable surfactants in this regard include well known non-ionic, anionic, cationic or zwitterionic surfactants, such as the Tween series, e.g. Tween 80. Alternatively, cores may be subjected to a preparatory surface treatment if the active ingredient that is employed as part of (or as) that core is susceptible to reaction with one or more precursor compounds that may be present in the gas phase during the coating (e.g. the ALD) process.

Application of ‘intermediary’ layers/surface treatments of this nature may alternatively be achieved by way of a liquid phase non-coating technique, followed by a lyophilisation, spray drying or other drying method, to provide particles with surface layers to which coating materials may be subsequently applied.

Outer surfaces of particles of compositions made by the process of the invention may also be derivatized or functionalized, e.g. by attachment of one or more chemical compounds or moieties to the outer surfaces of the final layer of coating material, e.g. with a compound or moiety that enhances the targeted delivery of the particles within a patient to whom the nanoparticles are administered. Such a compound may be an organic molecule (such as PEG) polymer, an antibody or antibody fragment, or a receptor-binding protein or peptide, etc.

Alternatively, the moiety may be an anchoring group such as a moiety comprising a silane function (see, for example, Herrera et al., J. Mater. Chem., 18, 3650 (2008) and U.S. Pat. No. 8,097,742). Another compound, e.g. a desired targeting compound may be attached to such an anchoring group by way of covalent bonding, or non-covalent bonding, including hydrogen bonding, or van der Waals bonding, or a combination thereof.

The presence of such anchoring groups may provide a versatile tool for targeted delivery to specific sites in the body. Alternatively, the use of compounds such as PEG may cause particles to circulate for a longer duration in the blood stream, ensuring that they do not become accumulated in the liver or the spleen (the natural mechanism by which the body eliminates particles, which may prevent delivery to diseased tissue).

Compositions made by the process of the invention are either suitable for administration to patients as they are prepared (i.e. as a plurality of particles) or are preferably formulated together with one or more pharmaceutically-acceptable excipients, including adjuvants, diluents or carriers for use in the medicinal or veterinary fields (including in therapy and/or, if the core comprises a diagnostic material, in diagnostics).

There is further provided compositions made by the process of the invention for use in medicine, diagnostics, and/or in veterinary practice and a pharmaceutical (or veterinary) formulation comprising a composition made by a process of the invention and a pharmaceutically-(or veterinarily-) acceptable adjuvant, diluent or carrier.

Compositions made by the process of the invention may be administered locally, topically or systemically, for example orally (enterally), by injection or infusion, intravenously or intraarterially (including by intravascular or other perivascular devices/dosage forms (e.g. stents)), intramuscularly, intraosseously, intracerebrally, intracerebroventricularly, intrasynovially, intrasternally, intrathecally, intralesionally, intracranially, intratumorally, cutaneously, intracutaneous, subcutaneously, transmucosally (e.g. sublingually or buccally), rectally, transdermally, nasally, pulmonarily (e.g. by inhalation, tracheally or bronchially), topically, or by any other parenteral route, such as subcutaneously or intramuscularly, optionally in the form of a pharmaceutical (or veterinary) preparation comprising the compound in a pharmaceutically (or veterinarily) acceptable dosage form.

The incorporation of compositions made by the process of the invention into pharmaceutical formulations may be achieved with due regard to the intended route of administration and standard pharmaceutical practice. Pharmaceutically acceptable excipients, such as carriers may be chemically inert to the biologically-active agent and may have no detrimental side effects or toxicity under the conditions of use. Such pharmaceutically acceptable carriers may also impart an immediate, or a modified, release of active agent from particles in compositions made by way of the process of the invention.

Pharmaceutical (or veterinary) formulations comprising compositions made by the process of the invention may include particles of different types, for example particles comprising different active ingredients, comprising different functionalization (as described hereinbefore), particles of different sizes, and/or different thicknesses of the layers of coating materials, or a combination thereof. By combining, in a single pharmaceutical formulation, particles with different coating thicknesses and/or different core sizes, the drug release following administration to patient may be controlled (e.g. varied or extended) over a specific time period.

For peroral administration (i.e. administration to the gastrointestinal tract by mouth with swallowing), compositions made by the process of the invention may be formulated in a variety of dosage forms, Pharmaceutically acceptable carriers or diluents may be solid or liquid. Solid preparations include granules (in which granules may comprise some or all of the plurality of particles of a composition made by a process of the invention in the presence of e.g. a carrier and other excipients, such as a binder or pH adjusting agents), compressed tablets, pills, lozenges, capsules, cachets, etc. Carriers include materials that are well known to those skilled in the art, including those disclosed hereinbefore in relation to the formulation of biologically active agents within cores, as well as magnesium carbonate, pectin, dextrin, starch, gelatin, tragacanth, methylcellulose, sodium carboxymethylcellulose, a low melting wax, cocoa butter, lactose, microcrystalline cellulose, low-crystalline cellulose, and the like.

Solid dosage forms may comprise further excipients, such as flavouring agents, lubricants, binders, preservatives, disintegrants, and/or encapsulating materials. For example, compositions made by the process of the invention may be encapsulated e.g. in a soft or hard shell capsule, e.g. a gelatin capsule.

Compositions made by the process of the invention formulated for rectal administration may include suppositories that may contain, for example, a suitable non-irritating excipient, such as cocoa butter, synthetic glyceride esters or polyethylene glycols, which are solid at ordinary temperatures, but which liquefy and/or dissolve in the rectal cavity to release the particles of the compositions made by the process of the invention.

For parenteral administration, such as subcutaneous and/or intramuscular injections, the compositions made by the process of the invention may be in the form of sterile injectable and/or infusible dosage forms, for example, sterile aqueous or oleaginous suspensions of compositions made by the process of the invention.

Sterile aqueous suspensions of the particles of a composition made by the process of the invention may be formulated according to techniques known in the art. The aqueous media should contain at least about 50% water, but may also comprise other aqueous excipients, such as Ringer's solution, and may also include polar co-solvents (e.g. ethanol, glycerol, propylene glycol, 1,3-butanediol, polyethylene glycols of various molecular weights and tetraglycol); viscosity-increasing, or thickening, agents (e.g. carboxymethylcellulose, microcrystalline cellulose, hydroxypropylmethyl cellulose, hydroxyethyl cellulose, ethyl hydroxyethyl cellulose, sodium starch glycolate, Poloxamers, such as Poloxamer 407, polyvinylpyrrolidone, cyclodextrins, such as hydroxypropyl-β-cyclodextrin, polyvinylpyrrolidone and polyethylene glycols of various molecular weights); surfactant/wetting agents to achieve a homogenous suspension (e.g. sorbitan esters, sodium lauryl sulfate; monoglycerides, polyoxyethylene esters, polyoxyethylene alkyl ethers, polyoxylglycerides and, preferably, Tweens (Polysorbates), such as Tween 80 and Tween 20). Preferred ingredients include isotonicity-modifying agents (e.g. sodium lactate, dextrose and, especially, sodium chloride); pH adjusting and/or buffering agents (e.g. citric acid, sodium citrate, and especially phosphate buffers, such as disodium hydrogen phosphate dihydrate, sodium acid phosphate, sodium dihydrogen phosphate monohydrate and combinations thereof, which may be employed in combination with standard inorganic acids and bases, such as hydrochloric acid and sodium hydroxide); as well as other ingredients, such as mannitol, croscarmellose sodium and hyaluronic acid.

Oleaginous, or oil-based carrier systems may comprise one or more pharmaceutically- or veterinarily-acceptable liquid lipid, which may include fixed oils, such as mono-, di- or triglycerides, including miglyol (e.g. 812N), propylene glycol dicaprylocaprate (Miglyol 840, C8/C10 esters), tricaprylin (Miglyol oil), gelucire 43/01, kollisolv GTA, labrafil. The carrier systems may also comprise polysorbates, such as polysorbate 20, polysorbate 60, polysorbate 80, glycols, such as propylene glycol, polyethylene glycol, polyethylene glycol 300, polyethylene glycol 400, polyethylene glycol 600, and/or natural and/or refined pharmaceutically-acceptable oils, such as olive oil, peanut oil, soybean oil, corn oil, cottonseed oil, sesame oil, castor oil, oleic acid, and their polyoxyethylated versions (e.g. sorbitan trioleate, lauroglycol 90, capryol PGMC, PEG-60 hydrogenated castor oil, polyoxyl 35 castor oil). More preferred carrier systems include mono-, di- and/or triglycerides, wherein most preferred is medium chain triglycerides, such as alkyl chain triglycerides (e.g. C6-C12 alkyl chain triglycerides).

Such injectable suspensions may be formulated in accordance with techniques that are well known to those skilled in the art, by employing suitable dispersing or wetting agents (e.g. Tweens, such as Tween 80), and suspending agents.

Compositions made by the process of the invention suitable for injection may be in the form of a liquid, a sol, a paste, or a gel, administrable via a surgical administration apparatus, e.g. a syringe with a needle for injection, a catheter or the like, to form a depot formulation.

The use of compositions made by the process of the invention may control the dissolution rate and the pharmacokinetic profile by reducing any burst effect as hereinbefore defined and/or by reducing the Cmax in a plasma concentration-time profile, and thus increasing the length of release of biologically active ingredient from that formulation.

These factors not only reduce the frequency at or over which a formulation by way of the process of the invention needs to be administered to a subject, but also allows the subject more time as an out-patient, and so to have a better quality of life.

The compositions made by way of the process of the invention also has the advantage that by controlling the release of active ingredient at a steady rate over a prolonged period of time, a lower daily exposure to a potentially toxic drug is provided, which is expected to reduce unwanted side effects.

Compositions made by the process of the invention may be contained within a reservoir and an injection or infusion means, wherein coated particles and carrier systems are housed separately and in which admixing occurs prior to and/or during injection or Infusion.

Compositions made by the process of the invention may also be formulated for inhalation, e.g. as an inhalation powder for use with a dry powder inhaler (see, for example, those described by Kumaresan et al., Pharma Times, 44, 14 (2012) and Mack et al., Inhalation, 6, 16 (2012)), the relevant disclosures thereof are hereby incorporated by reference. Suitable particle sizes for the plurality of particles in a composition made by a process of the invention for use in inhalation to the lung are in the range of about 2 to about 10 μm.

Compositions made by the process of the invention may also be formulated for administration topically to the skin, or to a mucous membrane. For topical application, the pharmaceutical formulations may be provided in the form of e.g. a lotion, a gel, a paste, a tincture, a transdermal patch, a gel for transmucosal delivery, all of which may comprise a composition made by a process of the invention. The composition may also be formulated with a suitable ointment containing a composition made by a process of the invention suspended in a carrier, such as a mineral oil, liquid petroleum, white petroleum, propylene glycol, polyoxyethylene polyoxypropylene compound, emulsifying wax or water. Suitable carrier for lotions or creams include mineral oils, sorbitan monostearate, polysorbate 60, cetyl esters wax, cetaryl alcohol, 2-octyldodecanol, benzyl alcohol and water.

Pharmaceutical formulations may comprise between about 1% to about 99%, such as between about 10% (such as about 20%, e.g. about 50%) to about 90% by weight of the coated particles, with the remainder made up by carrier system and/or other pharmaceutically acceptable excipients.

Pharmaceutical formulations may be in the form of a liquid, a sol or a gel, which is administrable via a surgical administration apparatus, e.g. a needle, a catheter or the like, to form a depot formulation.

In any event, compositions made by the process of the invention, may be formulated with conventional pharmaceutical additives and/or excipients used in the art for the preparation of pharmaceutical formulations, and thereafter incorporated into various kinds of pharmaceutical preparations and/or dosage forms using standard techniques (see, for example, Lachman et al., ‘The Theory and Practice of Industrial Pharmacy’, Lea & Febiger, 3rd edition (1986); ‘Remington: The Science and Practice of Pharmacy’, Troy (ed.), University of the Sciences in Philadelphia, 21st edition (2006); and/or ‘Aulton's Pharmaceutics: The Design and Manufacture of Medicines’, Aulton and Taylor (eds.), Elsevier, 4th edition, 2013), and the documents referred to therein, the relevant disclosures in all of which documents are hereby incorporated by reference. Otherwise, the preparation of suitable formulations may be achieved non-inventively by the skilled person using routine techniques.

According to a further aspect of the invention there is provided a process for the preparation of a pharmaceutical or veterinary formulation which comprises mixing together the coated particles prepared as described herein with a pharmaceutically-acceptable or a veterinarily-acceptable adjuvant, diluent or carrier.

It is preferred that such formulations are injectable and/or infusible and therefore comprise one or more compositions made by the process of the invention suspended in a pharmaceutically-acceptable or a veterinarily-acceptable aqueous and/or oleaginous carrier.

There is further provided an injectable and/or infusible dosage form comprising a compositions made by the process of the invention wherein said composition is contained within a reservoir that is connected to, and/or is associated with, an injection or infusion means (e.g. a syringe with a needle for injection, a catheter or the like). In this respect, compositions made by the process of the invention can be stored prior to being loaded into a suitable injectable and/or infusible dosing means (e.g. a syringe with a needle for injection) or may even be prepared immediately prior to loading into such a dosing means.

There is thus further provided a kit of parts comprising:
(a) a composition made by the process of the invention; and
(b) a pharmaceutically-acceptable or a veterinarily-acceptable carrier system,
   as well as a kit of parts comprising a composition made by the process of the invention along with instructions to the end user to admix those particles with pharmaceutically-acceptable or a veterinarily-acceptable aqueous and/or oleaginous carrier system.

There is further provided a pre-loaded injectable and/or infusible dosage form as described herein above, but modified by comprising at least two chambers, within one of which chamber is located composition made by the process of the invention and within the other of which is located a pharmaceutically-acceptable or a veterinarily-acceptable carrier system, wherein admixing, giving rise to a suspension or otherwise, occurs prior to and/or during injection or infusion.

Wherever the word ‘about’ is employed herein, for example in the context of amounts (e.g. concentrations, dimensions (sizes and/or weights), time periods), relative amounts (percentage, weight ratios, atomic ratios, size ratios, aspect ratios, proportions, factor or fractions), relative humidities, lux, temperatures or pressures, it will be appreciated that such variables are approximate and as such may vary by ±15%, such as ±10%, for example ±5% and preferably ±2% (e.g. ±1%) from the numbers specified herein. This is the case even if such numbers are presented as percentages in the first place (for example ‘about 15%’ may mean ±15% about the number 10, which is anything between 8.5% and 11.5%).

Compositions made by the process of the invention allow for the formulation of a large diversity of pharmaceutically active compounds. Compositions made by the process of the invention may be used to treat effectively a wide variety of disorders depending on the biologically active agent that is included.

Compositions made by the process of the invention may further be formulated in the form of injectable suspension of coated particles with a size distribution that is both even and capable of forming a stable suspension within the injection liquid (i.e. without settling) and may be injected through a needle. In this respect, compositions made by the process of the invention may comprise an aqueous medium that comprises inactive ingredients that may prevent premature gelling of compositions made by the process of the invention, and or is viscous enough to prevent sedimentation, leading to suspensions that are not ‘homogeneous’ and thus the risk of under or overdosing of active ingredient.

Furthermore, compositions made by the process of the invention can be stored under normal storage conditions, and maintain their physical and/or chemical integrity.

The phrase ‘maintaining physical and chemical integrity’ essentially means chemical stability and physical stability.

By ‘chemical stability’, we include that any compositions made by the process of the invention may be stored (with or without appropriate pharmaceutical packaging), under normal storage conditions, with an insignificant degree of chemical degradation or decomposition.

By ‘physical stability’, we include that the any compositions made by the process of the invention may be stored (with or without appropriate pharmaceutical packaging), under normal storage conditions, with an insignificant degree of physical transformation, such as sedimentation as described above, or changes in the nature and/or integrity of the coated particles, for example in the coating itself or the active ingredient (including dissolution, solvatisation, solid state phase transition, etc.).

Examples of ‘normal storage conditions’ for compositions made by the process of the invention include temperatures of between about −50° C. and about +80° C. (preferably between about −25° C. and about +75° C., such as about 50° C.), and/or pressures of between about 0.1 and about 2 bars (preferably atmospheric pressure), and/or exposure to about 460 lux of UV/visible light, and/or relative humidities of between about 5 and about 95% (preferably about 10 to about 40%), for prolonged periods (i.e. greater than or equal to about twelve, such as about six months).

Under such conditions, compositions made by the process of the invention may be found to be less than about 15%, more preferably less than about 10%, and especially less than about 5%, chemically and/or physically degraded/decomposed, as appropriate. The skilled person will appreciate that the above-mentioned upper and lower limits for temperature and pressure represent extremes of normal storage conditions, and that certain combinations of these extremes will not be experienced during normal storage (e.g. a temperature of 50° C. and a pressure of 0.1 bar).

Furthermore, compositions made by the process of the invention may provide a release and/or pharmacokinetic profile that minimizes any burst effect and/or minimize Cmax, which is characterised by a concentration maximum shortly after administration.

The compositions and processes described herein may have the advantage that, in the treatment of a relevant condition with a particular biologically active agent, they may be more convenient for the physician and/or patient than, be more efficacious than, be less toxic than, have a broader range of activity than, be more potent than, produce fewer side effects than, or that it may have other useful pharmacological properties over, any similar treatments that may be described in the prior art for the same active ingredient.

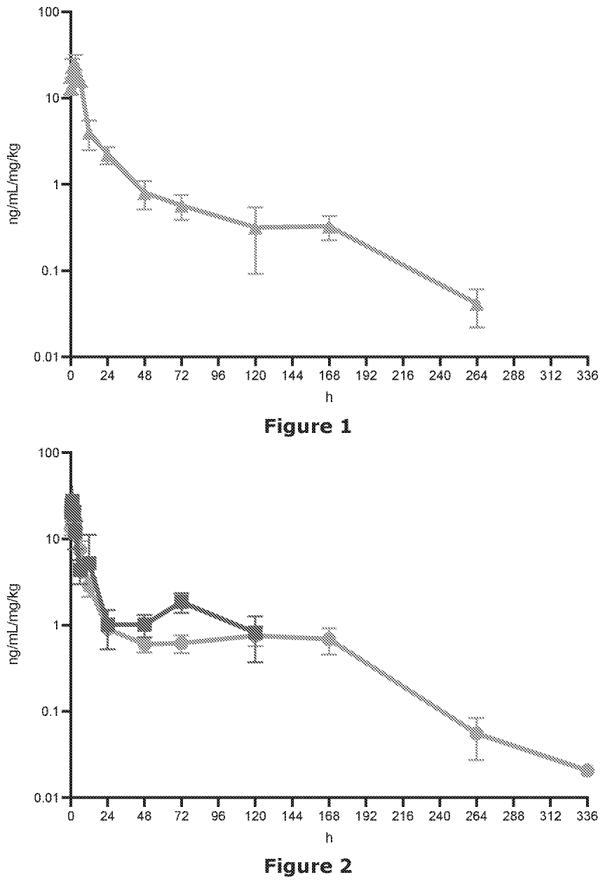

The invention is illustrated, but in no way limited, by the following examples with reference to the attached figures in which FIGS. 1 to 4 show dose adjusted plasma concentration vs time curves after administration of a sample prepared according to a respective example.

### EXAMPLES

### Comparative Example 1

### Coated Lenalidomide Microparticles Using Continuous Flow ALD I

Microparticles of lenalidomide 99.9% (APIChem, China) were used as received. The mean diameter of the lenalidomide particles was 10 μm as determined by laser diffraction (Shimadzu, SALD-7500nano, Kyoto, Japan). The particle size distribution, as determined by laser diffraction, was as follows: D₁₀ 2.7 μm; D₅₀ 10.4 μm and D₅₀ 24.9 μm.

The microparticles, or solid cores, were coated as described in steps 1-4.

1. The microparticles were loaded into an ALD reactor (Picosun, SUNALET R-series, Espoo, Finland) and subjected to three ALD cycles employing diethyl zinc and water as precursors, followed by one ALD cycle of trimethylaluminium and water as precursors at a reactor temperature of 50° C. This was repeated six times, i.e. 6×(3 cycles of Zn+1 cycle of Al), resulting in a total of 24 cycles. By this process, a first layer of mixed oxide with an atomic ratio of approximately 3:1 zinc:aluminium was formed.

The ALD reactor comprises a reaction chamber into which the microparticles were loaded. The ALD reactor further comprises precursor bottles containing each precursor separately, each precursor bottle being coupled to the reaction chamber via a valve. The ALD reactor also comprises a pump and associated piping for pumping an inert gas such as nitrogen through the reaction chamber, which pump is also coupled to the reaction chamber via a valve.

The ALD-cycle was performed as follows, wherein steps a to e represent a first cycle, subsequent cycles start from step d as specified in step l and the last cycle ends at step k:
a. Reagent pulse: Water was evaporated and carried into the reaction chamber by inert nitrogen gas by opening a valve to the precursor bottle for 0.1 s. Water adsorb to the surface of the drug particles, presenting hydroxyl groups on the exterior or the particle.
b. The reactor was thereafter pumped for 3 s.
c. Steps a-b above were repeated 100 times.
d. Purging pulse: The chamber was purged with nitrogen. Gaseous water, and organic gases in case this is not the first cycle, was removed.
e. Reagent pulse: Diethylzinc or trimethylaluminum was evaporated and carried into the reaction chamber by inert nitrogen gas by opening a valve to the precursor bottle for 0.1 s. Diethylzinc or trimethylaluminum adsorbs to the surface of the drug particles and reacts with hydroxyl groups. This releases ethane from diethylzinc or methane from trimethylaluminum.
f. The reactor was thereafter pumped for 3 s.
g. Steps e-f above were repeated 100 times.
h. Purging pulse: The chamber was purged with nitrogen. Non-reacted reagents and organic gases were removed.
i. Reagent pulse: Water was evaporated and carried into the reaction chamber by inert nitrogen gas by opening a valve to the precursor bottle for 0.1 s. Water adsorb to the surface of the drug particles and reacts with the metalloorganic surface. Remaining ethyl or methyl groups are converted to ethane and methane, respectively. The surface is thereby covered in a metal oxide layer, presenting hydroxyl groups on the exterior of the particle.
j. The reactor was thereafter pumped for 3 s.
k. Steps i-j above was repeated 100 times
l. The cycle was repeated from step d.

2. Next the powder was removed from the reactor and deagglomerated by means of sonic sifter (Tsutsui Sonic Agitated Sifting Machine SW-20AT) with a 32 μm mesh size sieve.

3. The resultant deagglomerated powder was re-loaded into the ALD reactor and steps 1-2 were repeated two times, forming a second and a third layer of mixed oxide with an atomic ratio of approximately 3:1 zinc:aluminium.

4. The resultant deagglomerated powder was re-loaded into the ALD reactor and step 1 was repeated one time.

### Comparative Example 2

### Coated Lenalidomide Microparticles Using Continuous Flow ALD II

This was similar to Comparative Example 1 except two additional layers were formed. In other words, step 3 was repeated once before step 4 was performed.

### Example 3

### Coated Lenalidomide Microparticles Using Stop-Flow ALD I

Microparticles of lenalidomide 99.9% (Kyongbo, South Korea) were used as received. The mean diameter of the lenalidomide particles was 4 μm as determined as described in Comparative Example 1 above. The particle size distribution, as determined by laser diffraction, was as follows: D₁₀ 0.6 μm; D₅₀ 3.9 μm and D₉₀ 15.6 μm.

The microparticles were coated essentially as described in steps 1˜4 of Comparative Example 1, except that the ALD-cycle was performed as follows (steps a-d represent the first cycle, subsequent cycles start from step a as specified in step e).
a. Reagent Pulse 1:
   i. The valve on the piping between the pump and the ALD reactor was closed.
   ii. The valve on the water precursor bottle was opened for 1 s letting evaporated water fill the reaction chamber.
   iii. The valve to the water precursor bottle was closed and, before opening the pump valve again, the chamber was rested for 30 s (soaking time) to ensure the water vapor adsorb to the surface of the drug particles, presenting hydroxyl groups on the exterior or the particles.
   iv. The reactor was thereafter pumped for 9 s.
   v. Steps i-iv above were repeated 20 times.
b. Purging pulse: The chamber was purged with nitrogen in a continuous flow. Gaseous water, and organic gases in case this is not the first cycle, was removed.
c. Reagent pulse:
   i. The valve on the piping between the pump and the ALD reactor was closed.
   ii. The valve on the diethylzinc or trimethylaluminium precursor bottle was opened for 1 s letting evaporated metal containing precursor fill the reaction chamber.
   iii. The valve to the precursor bottle was closed and, before opening to the pump again, the chamber was rested for 30 s (soaking time) to ensure the metal containing precursor vapor react with the hydroxyl groups on the surface of the drug particles,
   iv. The reactor was thereafter pumped for 9 s.
   v. Steps i-iv above was repeated 20 times.
d. Purging pulse: The chamber was purged with nitrogen in a continuous flow. Non-reacted reagents and organic gases were removed.
e. The cycle was repeated from step a.

### Example 4

### Coated Lenalidomide Microparticles Using Stop-Flow ALD II

This was similar to Comparative Example 3 but two additional layers were formed. In other words, step 3 was repeated once before step 4 was performed.

### Example 5

### Determination of Drug Load

The present Example describes the analysis of the drug load of lenalidomide of the particles obtained in Comparative Examples 1 and 2 and Examples 3 and 4.

### Material and Method

To determine the drug load (i.e. w/w % of lenalidomide in the powder), UPLC (Prominence-i (Shimadzu, Japan) equipped with a diode array detector (Shimadzu, Japan) set at 223 nm was employed using a 4.6×100 mm, 2.6 μm particles, C18WP column (SunShell, ChromaNik Technologies Inc, Osaka, Japan)) was used. The materials were dissolved in 2 M phosphoric acid in acetonitrile/water (1:1) and was diluted with methanol/12.5 mM phosphate buffer pH 3.4 (4:1) before filtration (0.2 μm RC, Lab Logistics Group, Germany) and further analyzed with UPLC (n=2).

The UPLC assay is set-up according to Table 1.

TABLE 1
Parameters for the UPLC-method.

| Column | SunShell C18WP 4.6*100 mm, 2.6 μm | |
|---|---|---|
| | particles, Nanexa column ID 1. | |
| Temperature, column oven | 20° C. | |
| Flow | 0.7 mL/min | |
| | Time (min) | % Mobile phase B |
| Gradient program | 0-0.5 | 10 |
| | 4 | 12 |
| | 12 | 32 |
| | 15 | 32 |
| | 17.50 | 10 |
| | 17.50-22 | 10 |

| | |
|---|---|
| Mobile phase A | 5 mM phosphate buffer pH 3.4 |
| Mobile phase B | Methanol |
| Wash solution | 50% Acetonitrile in water |
| Run time | 22 min |
| Temperature, autosampler | 25° C. |
| Injection volume | 5 μL |
| UV detection wavelength | 223 nm |

Concentrations of injected samples were calculated automatically by the software supplied by the manufacturer. Drug load was calculated according to the equation below, wherein CLEN is the analyzed concentration of lenalidomide and mₛₐₘₚₗₑ is sample mass:

$$Drugload ( \% ) = 100 \times \frac{{C}_{LEN} ( mg / L )}{\frac{{m}_{sample} ( mg )}{End Volume ( L )}}$$

### Results

The drug load lenalidomide of the particles according to Comparative Example 1 was determined as 94.8%.

The drug load lenalidomide of the particles according to Comparative Example 2 was determined as 92.6%.

The drug load lenalidomide of the particles according to Example 3 was determined as 76.0%.

The drug load lenalidomide of the particles according to Example 4 was determined as 67.7%.

### Example 6

### Coating Integrity

The present Example describes the analysis of the coating integrity of the particles obtained in Comparative Examples 1 and 2 and Examples 3 and 4.

The coating integrity of the coated lenalidomide was determined by preparing a suspension of the coated material in DMSO, a solvent which dissolves the API but not the coating material. Therefore, release of lenalidomide from the product can only happen due to “defects” in coating. By measuring the released lenalidomide with an HPLC method, the integrity of the coating can be assessed. The lower the percentage of the released lenalidomide the better the coating integrity.

### Material and Method

To determine the coating integrity (i.e. w/w % of lenalidomide released in the powder), UPLC (Prominence-i (Shimadzu, Japan) equipped with a diode array detector (Shimadzu, Japan) set at 223 nm was employed using a 4.6×100 mm, 2.6 μm particles, C18WP column (SunShell, ChromaNik Technologies Inc, Osaka, Japan)) was used. The 25 mg of the materials were dispersed in 25 mL DMSO and put on a turning table for 3 h. A 1 mL sample was withdrawn for filtration (0.2 μm RC, Lab Logistics Group, Germany) and further analysis with UPLC (n=1) according to set-up in Table 1 above.

Concentrations of injected samples were calculated automatically by the software supplied by the manufacturer. The amount of API dissolved in the coating integrity assay was calculated according to the equation below, wherein cₐ is the concentration of API from analysis (mg/ml), Vₜ is the total sample volume, (ml), man is the mass of API weighed in (mg), and drug load is the API content of the coated API (fraction):

$$Amount API dissolved ( \% ) = 100 \frac{{c}_{α} \cdot {V}_{t}}{{m}_{API} \cdot drugload}$$

### Results

The amount of API dissolved in the coating integrity assay of the particles according to Comparative Example 1 was determined as 74%.

The amount of API dissolved in the coating integrity assay of the particles according to Comparative Example 2 was determined as 51%.

The amount of API dissolved in the coating integrity assay of the particles according to Example 3 was determined as 15%.

The amount of API dissolved in the coating integrity assay of the particles according to Example 4 was determined as 7%.

### Example 7

### Preparation of Formulations

The present Example describes the preparation of formulations of the coated lenalidomide particles as disclosed herein.

### Material and Method

Suspensions of coated microparticles of lenalidomide according to Comparative Examples 1 and 2 and Examples 3 and 4 was prepared.

The powder of coated microparticles of lenalidomide according to Comparative Examples 1 and 2 and Examples 3 and 4 was mixed together with Hyonate® vet (Boehringer Ingelheim Animal Health, France) as vehicle. Hyonate® vet is a veterinary medicinal product used in injecting animals comprising a sterile, isotonic, phosphate buffered solution of 10 mg/mL of sodium hyaluronate (pH 7.4). The composition of Hyonate® vet is a presented in Table 2 below.

TABLE 2
Composition of the vehicle for suspension
of particles as disclosed herein.

| | | | |
|---|---|---|---|
| Sodium hyaluronate | 10 | mg | Active substance |
| Sodium chloride | 8.5 | mg | Isotonic agent |
| Sodium acid phosphate | 0.223 | mg | Buffering agent |
| Sodium dihydrogen | 40 | μg | Buffering agent |

| | | |
|---|---|---|
| phosphate monohydrate | | |
| Hydrochloric acid | q.s. | For pH adjustment |
| Sodium hydroxide | q.s. | For pH adjustment |
| Water for injection | q.s. ad 1 mL | Solvent |

The microparticles were reconstituted by adding the vehicle to a vial with powder of microparticles to obtain a lenalidomide concentration of:
50 mg/ml lenalidomide of the particles according to Comparative Example 1,
5 mg/ml and 50 mg/ml lenalidomide of the particles according to Comparative Example 2,
20 mg/ml lenalidomide of the particles according to Example 3, and
20 mg/ml lenalidomide of the particles according to Example 4.

### Results

Suspensions of coated lenalidomide particles according to Comparative Examples 1 and 2 and Examples 3 and 4 respectively, were obtained.

### Comparative Example 8

### In Vivo Pharmacokinetic Study I

The present Example describes a preclinical pharmacokinetic study of lenalidomide following administration of coated lenalidomide formulations according to Comparative Examples 1 and 2 in male and female Sprague Dawley rats,

### Material and Method

Male and Female Sprague Dawley rats weighing between 231 and 285 g at the day of administration were supplied by Charles River (UK). The duration of the study was 14 days. 36 rats were used in the study.

The administration areas were clipped free from hair prior to injection and the injection site will be marked. A suspension prepared as described in Example 7 with coated lenalidomide particles according to Comparative Example 1 and 2 were drawn into a 1 mL BD syringe and single, subcutaneous injections (ca. 0.12 mL) was administered through a 23 G needle (BD microlance) into the flank of each rat.

Blood samples (ca 0.2 mL) were be collected from the tail vein into K₂EDTA tubes at the following time-points: 0.25, 0.5, 1, 2, 3, 6, 12, 24, 48, 72, 120, 168, 264, and 336 h post-dose. As soon as practically possible following blood sampling, plasma was separated by centrifugation (1500 g for 10 min at 4° C.). Animals were sacrificed on the last day of the study. Plasma samples were stored in a freezer at −80° C. pending analysis.

To determine the plasma concentration, UPLC-MS/MS was used.

Samples were prepared by pipetting 35 μL of rat plasma into a 96 well plate, adding 35 μL 5% DMF in acetonitrile, 70 μL of an internal standard working solution using the TECAN Genesis liquid handling robot. The 96 well plates were shaken for 15 minutes and centrifuged. All samples were then injected on a UPLC-MS/MS system (Xevo TQ-s micro coupled to an Acquity I-Class UPLC system, Waters, Milford, MA, USA) with the set-up as specified in Table 3 below.

TABLE 3
Parameters for the UPLC-MS/MS-method.

| | |
|---|---|
| Column | Acquity CSH C18 (50 × 2.1 mm, 1.7 μm) |
| Mobile phase A | 0.10% Formic Acid in Water |
| Mobile phase B | ACN:DMSO (95:5) |
| Wash solution | ACN:MeOH (1:1) |

Pharmacokinetic analysis of lenalidomide in plasma was performed according to standard non-compartmental approach using PKanalix (2021, Lixoft, Antony, France), Dose-normalised plasma concentrations of lenalidomide after single subcutaneous administration of the formulation was assessed and the following plasma pharmacokinetic parameters was evaluated:
Dose will be expressed in mg/kg body weight of the rat.
‘Cₘₐₓ’: the maximum concentration found in analysis expressed in ng/mL.
‘AUC_{∞}’: the area under concentration vs. time curve up to infinite time expressed in ng*h/mL.
‘Cₘₐₓ/D’: the maximum concentration normalized to 1 mg/kg expressed in ng/ml/mg/kg body weight of the rat.
‘AUC_{∞}/D’: the area under concentration vs. time curve up to infinite time normalized to 1 mg/kg expressed in ng*h/mL/mg/kg body weight of the rat.
‘Fr. Rel.₀₋₁₂ₕ’: the fraction released during the first twelve hours of the area under concentration vs, time curve up to infinite time expressed as a percentage.

### Results

FIGS. 1 and 2 show the respective plasma concentration time curves (ng/ml plasma concentration lenalidomide versus h sampling time) for samples obtained by Comparative Examples 1 and 2, respectively.

TABLE 4
Results of plasma pharmacokinetic parameters evaluation.

| | Comparative | Comparative | Comparative |
|---|---|---|---|
| Parameter | Example 1 | Example 2 | Example 2 |
| Dose | 50 | 5 | 50 |
| Cₘₐₓ | 1250 | 142 | 986 |
| AUC_{∞} | 17300 | 712 | 14000 |
| AUC_{0-12 h} | 12000 | 650 | 6974 |
| Cₘₐₓ/D | 25.0 | 28.4 | 19.7 |
| AUC_{∞}/D | 346 | 142 | 280 |
| AUC_{0-12 h}/D | 240 | 130 | 139 |
| Fr. Rel._{0-12 h} | 0.694 | 0.913 | 0.498 |

### Example 9

### In Vivo Pharmacokinetic Study II

The present Example describes a preclinical pharmacokinetic study of lenalidomide following administration of coated lenalidomide formulations according to Examples 3 and 4 in male and female Sprague Dawley rats.

### Material and Method

Male Sprague Dawley rats weighing between 260 and 343 g at the day of administration were supplied by Charles River (UK). The duration of the study was 14 days. 44 rats were used in the study.

The administration areas were clipped free from hair prior to injection and the injection site will be marked. A suspension prepared as described in Example 7 with coated lenalidomide particles according to Comparative Example 3 and 4 were drawn into a 1 mL BD syringe and single, subcutaneous injections (ca. 0.15 mL) was administered through a 23 G needle (BD microlance) into the flank of each rat.

Blood samples (ca 0.2 mL) will be collected from the tail vein into K₂EDTA tubes at the following time-points: 0.25, 0.5, 1, 2, 3, 6, 12, 24, 48, 72, 120, 168, 216, 264, and 336 h post-dose. As soon as practically possible following blood sampling, plasma was separated by centrifugation (1500 g for 10 min at 4° C.). Animals were sacrificed on the last day of the study. Plasma samples were stored in a freezer at −80° C. pending analysis.

To determine the plasma concentration, UPLC-MS/MS was used.

Samples were prepared by pipetting 35 μL of rat plasma into a 96 well plate, adding 35 μL 5% DMF in acetonitrile, 70 μL of an internal standard working solution using the TECAN Genesis liquid handling robot. The 96 well plates were shaken for 15 minutes and centrifuged. All samples were then injected on a UPLC-MS/MS system (Xevo TQ-s micro coupled to an Acquity I-Class UPLC system, Waters, Milford, MA, USA) with the set-up as specified in Table 2 above.

Pharmacokinetic analysis of lenalidomide in plasma was performed according to standard non-compartmental approach using Phoenix WinNonlin, version 8.3 (Certara, USA), Dose-normalised plasma concentrations of lenalidomide after single subcutaneous administration of the formulation was assessed and the following plasma pharmacokinetic parameters was evaluated:
Dose will be expressed in mg/kg body weight of the rat.
‘Cₘₐₓ’: the maximum concentration found in analysis expressed in ng/ml.
‘AUC_{∞}’: the area under concentration vs. time curve up to infinite time expressed in ng*h/mL.
‘Cₘₐₓ/D’: the maximum concentration normalized to 1 mg/kg expressed in ng/ml/mg/kg body weight of the rat.
‘AUC_{∞}/D’: the area under concentration vs. time curve up to infinite time normalized to 1 mg/kg expressed in ng*h/mL/mg/kg body weight of the rat.
‘Fr. Rel.₀₋₁₂ₕ’: the fraction released during the first twelve hours of the area under concentration vs. time curve up to infinite time expressed as a percentage.

### Results

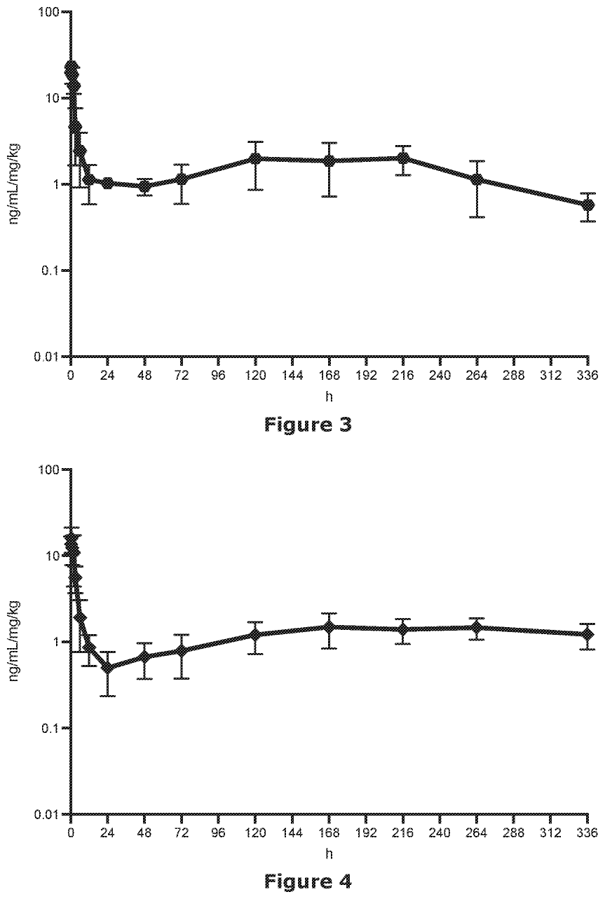

FIGS. 3 and 4 show the respective plasma concentration time curves (ng/ml plasma concentration lenalidomide versus h sampling time) for samples obtained by Examples 3 and 4, respectively,

TABLE 5
Results of plasma pharmacokinetic parameters evaluation.

| Example | Example 3 | Example 4 |
|---|---|---|
| Dose | 10 | 10 |
| Cₘₐₓ | 239 | 152 |
| AUC_{∞} | 7693 | 9279 |
| AUC_{0-12 h} | 668 | 452 |
| Cₘₐₓ/D | 23.9 | 15.2 |
| AUC_{∞}/D | 769.3 | 927.9 |
| AUC_{0-12 h}/D | 66.8 | 45.2 |
| Fr. Rel._{0-12 h} | 0.087 | 0.049 |

The dose normalized maximum concentration was lower for Example 3 compared with Comparative Example 1 indicated by both a lower maximum concentration and a lower fraction released during the first twelve hours.

The dose normalized maximum concentration was lower for Example 4 compared with Comparative Example 2 indicated by both a lower maximum concentration and a lower fraction released during the first twelve hours.

Altogether, Comparative Example 8 and Example 9 demonstrated that the coated lenalidomide particles produced by stop-flow ALD showed a release profile with lower initial release than the coated lenalidomide particles produced using continuous flow ALD.

### Comparative Example 10

### Coated Indomethacin Microparticles Using Continuous Flow ALD I

Microparticles of indomethacin 99.9% (ReechPharma, CA, USA) were used as received. The mean diameter of the indomethacin particles was 10.5 μm as determined by laser diffraction (Shimadzu, SALD-7500nano, Kyoto, Japan). The particle size distribution, as determined by laser diffraction, was as follows: D₁₀ 4.2 μm; D₅₀ 10.5 μm and D₉₀ 23.8 μm.

The microparticles, or solid cores, were coated as described in steps 1-4.

1. The microparticles were loaded into an ALD reactor (Picosun, SUNALE™ R-series, Espoo, Finland) and subjected to three ALD cycles employing diethyl zinc and water as precursors, followed by one ALD cycle of trimethylaluminium and water as precursors at a reactor temperature of 50° C. This was repeated six times, i.e. 6×(3 cycles of Zn+1 cycle of Al), resulting in a total of 24 cycles, By this process, a first layer of mixed oxide with an atomic ratio of approximately 3:1 zinc:aluminium was formed.

The ALD reactor comprises a reaction chamber into which the microparticles were loaded. The ALD reactor further comprises precursor bottles containing each precursor separately, each precursor bottle being coupled to the reaction chamber via a valve. The ALD reactor also comprises a pump and associated piping for pumping an inert gas such as nitrogen through the reaction chamber, which pump is also coupled to the reaction chamber via a valve.

The ALD-cycle was performed as follows, wherein steps a to e represent a first cycle, subsequent cycles start from step d as specified in step l and the last cycle ends at step k:
a. Reagent pulse: Water was evaporated and carried into the reaction chamber by inert nitrogen gas by opening a valve to the precursor bottle for 0.1 s. Water adsorb to the surface of the drug particles, presenting hydroxyl groups on the exterior or the particle.
b. The reactor was thereafter pumped for 3 s.
c. Steps a-b above were repeated 100 times.
d. Purging pulse: The chamber was purged with nitrogen. Gaseous water, and organic gases in case this is not the first cycle, was removed.
e. Reagent pulse: Diethylzinc or trimethylaluminum was evaporated and carried into the reaction chamber by inert nitrogen gas by opening a valve to the precursor bottle for 0.1 s. Diethylzinc or trimethylaluminum adsorbs to the surface of the drug particles and reacts with hydroxyl groups. This releases ethane from diethylzinc or methane from trimethylaluminum.
f. The reactor was thereafter pumped for 3 s.
g. Steps e-f above were repeated 100 times.
h. Purging pulse: The chamber was purged with nitrogen. Non-reacted reagents and organic gases were removed.
i. Reagent pulse: Water was evaporated and carried into the reaction chamber by inert nitrogen gas by opening a valve to the precursor bottle for 0.1 s. Water adsorb to the surface of the drug particles and reacts with the metalloorganic surface. Remaining ethyl or methyl groups are converted to ethane and methane, respectively. The surface is thereby covered in a metal oxide layer, presenting hydroxyl groups on the exterior of the particle.
j. The reactor was thereafter pumped for 3 s.
k. Steps i-j above was repeated 100 times
l. The cycle was repeated from step d.

2. Next the powder was removed from the reactor and deagglomerated by means of sonic sifter (Tsutsui Sonic Agitated Sifting Machine SW-20AT) with a 20 μm mesh size sieve.

3. The resultant deagglomerated powder was re-loaded into the ALD reactor and steps 1-2 were repeated two times, forming a second and a third layer of mixed oxide with an atomic ratio of approximately 3:1 zinc:aluminium.

4. The resultant deagglomerated powder was re-loaded into the ALD reactor and step 1 was repeated one time.

### Example 11

### Coated Indomethacin Microparticles Using Stop-Flow I

Microparticles of indomethacin 99.9% (ReechPharma, CA, USA) were used as received. The mean diameter and particle size distribution was as described in Comparative Example 10.

The microparticles were coated essentially as described in steps 1˜4 of Comparative Example 10, except that the ALD-cycle was performed as follows (steps a-d represent the first cycle, subsequent cycles start from step a as specified in step e).
a. Reagent pulse 1:
   i. The valve on the piping between the pump and the ALD reactor was closed.
   ii. The valve on the water precursor bottle was opened for 1 s letting evaporated water fill the reaction chamber.
   ill. The valve to the water precursor bottle was closed and, before opening the pump valve again, the chamber was rested for 15 s (soaking time) to ensure the water vapor adsorb to the surface of the drug particles, presenting hydroxyl groups on the exterior or the particles,
   iv. The reactor was thereafter pumped for 9 s.
   v. Steps i-iv above were repeated 20 times.
b. Purging pulse: The chamber was purged with nitrogen in a continuous flow, Gaseous water, and organic gases in case this is not the first cycle, was removed.
c. Reagent pulse:
   i. The valve on the piping between the pump and the ALD reactor was closed.
   ii. The valve on the diethylzinc or trimethylaluminium precursor bottle was opened for 1 s letting evaporated metal containing precursor fill the reaction chamber.
   iii. The valve to the precursor bottle was closed and, before opening to the pump again, the chamber was rested for 15 s (soaking time) to ensure the metal containing precursor vapor react with the hydroxyl groups on the surface of the drug particles.
   iv. The reactor was thereafter pumped for 9 s.
   v. Steps i-iv above was repeated 20 times.
d. Purging pulse: The chamber was purged with nitrogen in a continuous flow. Non-reacted reagents and organic gases were removed.
e. The cycle was repeated from step a.

### Example 12

### Determination of Drug Load

The present Example describes the analysis of the drug load of indomethacin of the particles obtained in Example 11.

### Material and Method

To determine the drug load (i.e. w/w % of indomethacin in the powder), UPLC (Nexera, Shimadzu, Japan) equipped with a diode array detector (Shimadzu, Japan) set at 254 nm was employed using a 3×100 mm, 2.6 μm particles, phenyl-hexyl column (Thermo Fisher Scientific Inc., MA, USA) was used. The materials were dissolved in 2 M phosphoric acid in acetonitrile/water (1:1) and was diluted with acetonitrile/water (3:1) before filtration (0.2 μm RC, Lab Logistics Group, Germany) and further analyzed with UPLC (n=3).

The UPLC assay is set-up according to Table 6.

TABLE 6
Parameters for the UPLC-method.

| Column | Accucore Phenyl-Hexyl 3 × 100 mm, 2.6 μm | |
|---|---|---|
| | particles, Nanexa column ID 2C. | |
| Temperature, column oven | 40° C. | |
| Flow | 0.1 mL/min | |
| | Time (min) | % Mobile phase B |
| Gradient program | 0-2 | 30 |
| | 2-8 | 30-40 (linear) |
| | 8-11 | 40-70 (linear) |
| | 11-13 | 70 |
| | 13-13.1 | 70-30 (linear) |
| | 13.1-15 | 30 |

| | |
|---|---|
| Mobile phase A | Glacial acetic acid 10% (v/v) in water |
| Mobile phase B | Acetonitrile |
| Wash solution | 80% Acetonitrile in water |
| Run time | 15 min |
| Temperature, autosampler | 20° C. |
| Injection volume | 10 μL |
| UV detection wavelength | 254 nm |

Concentrations of injected samples were calculated automatically by the software supplied by the manufacturer. Drug load was calculated according to the equation below, wherein Cₛₐₘₚₗₑ is the analyzed concentration of indomethacin, Vₜ is the total sample volume and mₛₐₘₚₗₑ is sample mass:

$$Drug load ( \% ) = 100 \cdot ( {C}_{sample} \cdot {V}_{t} ) / {m}_{sample}$$

The drug load indomethacin of the particles according to Example 11 was determined as 83.2%.

### Example 13

### Determination of Coating Integrity

The present Example describes the analysis of the coating integrity of the particles obtained in Example 11.

The coating integrity of the coated indomethacin was determined by preparing a suspension of the coated material in DMSO, a solvent which dissolves the API but not the coating material. Therefore, release of indomethacin from the product can only happen due to “defects” in coating. By measuring the released indomethacin with an HPLC method, the integrity of the coating can be assessed. The lower the percentage of the released indomethacin the better the coating integrity.

### Material and Method

To determine the coating integrity (i.e. w/w % of indomethacin released in the powder), UPLC (Nexera, Shimadzu, Japan) equipped with a diode array detector (Shimadzu, Japan) set at 254 nm was employed using a 3×100 mm, 2.6 μm particles, phenyl-hexyl column (Thermo Fisher Scientific Inc., MA, USA) was used. The 25 mg of the materials were dispersed in 25 mL DMSO and put on a turning table for 3 h. A 1 mL sample was withdrawn for filtration (0.2 μm RC, Lab Logistics Group, Germany) and further analysis with UPLC (n=1) according to set-up in Table 6 above.

The concentrations of injected samples were calculated automatically by the software supplied by the manufacturer. The amount of API dissolved in the coating integrity assay was calculated according to the equation below, wherein ca is the concentration of API from analysis (mg/ml), Vₜ is the total sample volume (ml), m_{API} is the mass of API weighed in (mg), and drug load is the API content of the coated API (fraction):

$$Amount API dissolved ( \% ) = 100 \cdot ( {c}_{a} \cdot {V}_{t} ) / ( {m}_{API} \cdot drug load )$$

### Results

The amount of API dissolved in the coating integrity assay of the particles according to Example 11 was determined as 13.0%.

### Example 14

### Coated Indomethacin Microparticles Using Stop-Flow II

Microparticles of indomethacin 99.9% (ReechPharma, CA, USA) were used as received. The mean diameter and particle size distribution was as described in Comparative Example 10.

The microparticles were coated essentially as described in steps 1˜4 of Example 11, except that the soaking time was 30 s, rather than 15 s, in both steps a and c.

### Example 15

### Determination of Drug Load

The present Example describes the analysis of the drug load of indomethacin of the particles obtained in Example 14.

### Material and Method

The material and method was as described in Example 12 except that the particles analysed were those obtained in Example 14, rather than those obtained in Example 11.

### Results

The drug load indomethacin of the particles according to Example 14 was determined as 82.3%.

### Example 16

### Determination of Coating Integrity

The present Example describes the analysis of the coating integrity of the particles obtained in Example 14.

The coating integrity of the coated indomethacin was determined as described in Example 13.

### Material and Method

The material and method was as described in Example 13 except that the particles analysed were those obtained in Example 14, rather than those obtained in Example 11.

### Results

The amount of API dissolved in the coating integrity assay of the particles according to the Example 14 was determined as 10.6%.

### Comparative Example 17

### Coated Lenalidomide Microparticles Using Continuous Flow ALD

This was similar to Comparative Example 10 except microparticles of lenalidomide 99.9% (Kyongbo, South Korea) were used as received, rather than indomethacin 99.9%. The mean diameter of the lenalidomide particles was 4.3 μm as determined by laser diffraction (Shimadzu, SALD-7500nano, Kyoto, Japan). The particle size distribution, as determined by laser diffraction, was as follows: D10 0.9 μm; D50 4.3 μm and D90 15.6 μm.

Apart from the microparticles used, all other aspects of the process used in Comparative Example 17 were identical to those of Comparative Example 10.

### Example 18

### Coated Lenalidomide Microparticles Using Stop-Flow I

Microparticles of lenalidomide 99.9% (Kyongbo, South Korea) were used as received. The mean diameter of the lenalidomide particles was 4.3 μm as determined as described in Comparative Example 17 above. The particle size distribution, as determined by laser diffraction, was as follows: D10 0.9 μm; D50 4.3 μm and D90 15.6 μm.

The microparticles were coated essentially as described in steps 1˜4 of Comparative Example 17, except that:
in step 3, steps 1-2 were repeated four times, forming a second, third, fourth and fifth layer of mixed oxide with an atomic ratio of approximately 3:1 zinc:aluminium; and
the ALD-cycle was performed as follows (steps a-d represent the first cycle, subsequent cycles start from step a as specified in step e).
a. Reagent pulse 1:
   i. The valve on the piping between the pump and the ALD reactor was closed,
   ii. The valve on the water precursor bottle was opened for 1 s letting evaporated water fill the reaction chamber.
   iii. The valve to the water precursor bottle was closed and, before opening the pump valve again, the chamber was rested for 30 s (soaking time) to ensure the water vapor adsorb to the surface of the drug particles, presenting hydroxyl groups on the exterior or the particles.
   iv. The reactor was thereafter pumped for 9 s.
   v. Steps i-iv above were repeated 20 times.
b. Purging pulse: The chamber was purged with nitrogen in a continuous flow. Gaseous water, and organic gases in case this is not the first cycle, was removed.
c. Reagent pulse:
   i. The valve on the piping between the pump and the ALD reactor was closed.
   ii. The valve on the diethylzinc or trimethylaluminium precursor bottle was opened for 1 s letting evaporated metal containing precursor fill the reaction chamber.
   iii. The valve to the precursor bottle was closed and, before opening to the pump again, the chamber was rested for 30 s (soaking time) to ensure the metal containing precursor vapor react with the hydroxyl groups on the surface of the drug particles,
   iv. The reactor was thereafter pumped for 9 s.
   v. Steps i-iv above was repeated 20 times.
d. Purging pulse: The chamber was purged with nitrogen in a continuous flow. Non-reacted reagents and organic gases were removed.
e. The cycle was repeated from step a.

### Example 19

### Determination of Drug Load

The present Example describes the analysis of the drug load of lenalidomide of the particles obtained in Example 18.

### Material and Method

The material and method was as described in Example 12 except that the particles analysed were those obtained in Example 18, rather than those obtained in Example 11.

### Results

The drug load lenalidomide of the particles according to Example 18 was determined as 69.2%.

### Example 20

### Determination of Coating Integrity

The present Example describes the analysis of the coating Integrity of the particles obtained in Example 18.

The coating integrity of the coated lenalidomide was determined as described in Example 13.

### Material and Method

The material and method was as described in Example 13 except that the particles analysed were those obtained in Example 18, rather than those obtained in Example 11.

### Results

The amount of API dissolved in the coating integrity assay of the particles according to the Example 18 was determined as 6.8%.

### Example 21

### Preparation of Formulations

The present Example describes the preparation of formulations of the coated lenalidomide particles as disclosed herein.

The powder of coated microparticles of lenalidomide according to the Example was mixed together with Hyonate® vet (Boehringer Ingelheim Animal Health, France) as vehicle. Hyonate® vet is a veterinary medicinal product used in Injecting animals comprising a sterile, isotonic, phosphate buffered solution of 10 mg/ml of sodium hyaluronate (pH 7.4), The composition of Hyonate® vet is presented in Table 9 below.

TABLE 9
Composition of the vehicle for suspension
of particles as disclosed herein.

| | | | |
|---|---|---|---|
| Sodium hyaluronate | 10 | mg | Active substance |
| Sodium chloride | 8.5 | mg | Isotonic agent |
| Sodium acid phosphate | 0.223 | mg | Buffering agent |
| Sodium dihydrogen | 40 | μg | Buffering agent |

| | | |
|---|---|---|
| phosphate monohydrate | | |
| Hydrochloric acid | q.s. | For pH adjustment |
| Sodium hydroxide | q.s. | For pH adjustment |
| Water for injection | q.s. ad 1 mL | Solvent |

The microparticles were reconstituted by adding the vehicle to a vial with powder of microparticles to obtain a lenalidomide concentration of:
20 mg/mL lenalidomide of the particles according to Example 18.

### Example 22

### In Vivo Pharmacokinetic Study

The present Example describes a preclinical pharmacokinetic study of lenalidomide following administration of coated lenalidomide formulations according to Example 21 In male and female Sprague Dawley rats.

### Material and Method

Male and Female Sprague Dawley rats weighing between 260 and 343 g at the day of administration were supplied by Charles River (UK). The duration of the study was 14 days. In each study group of the study, 3 rats of each sex were used. The study groups were the coated microparticle formulation according to Example 21, and as control, uncoated microparticles of lenalidomide.

The administration areas were clipped free from hair prior to injection and the injection site will be marked. A suspension prepared as described in Example 21 with coated lenalidomide particles according to Example 18 were drawn into a 1 mL BD syringe and single, subcutaneous injections (ca. 0.12 mL) was administered through a 23 G needle (BD microlance) into the flank of each rat.

Blood samples (ca 0.2 mL) were be collected from the tail vein into K₂EDTA tubes at the following time-points: 0.25, 0.5, 1, 2, 3, 6, 12, 24, 48, 72, 120, 168, 264, and 336 h post-dose. As soon as practically possible following blood sampling, plasma was separated by centrifugation (1500 g for 10 min at 4° C.). Animals were sacrificed on the last day of the study. Plasma samples were stored in a freezer at −80° C. pending analysis.

To determine the plasma concentration, UPLC-MS/MS was used.

Samples were prepared by pipetting 35 μL of rat plasma into a 384 well plate, adding 35 μL 5% DMSO in acetonitrile, and 70 μL of an internal standard working solution, using a TECAN EVO Ware liquid handling robot. The 384 well plates were shaken for 15 minutes and centrifuged. All samples were then injected on a UPLC-MS/MS system (Xevo TQ-s micro coupled to an Acquity I-Class UPLC system, Waters, Milford, MA, USA) with the set-up as specified in Table 10 below.

TABLE 10
Parameters for the UPLC-MS/MS-method.

| | |
|---|---|
| Column | Acquity CSH C18 (50 × 2.1 mm, 1.7 μm) |
| Mobile phase A | 0.10% Formic Acid in Water |
| Mobile phase B | ACN:DMSO (95:5) |
| Wash solution | ACN:MeOH (1:1) |

Pharmacokinetic analysis of lenalidomide in plasma was performed according to using non-compartmental analysis (NCA) utilizing the software Phoenix WinNonlin, version 8.3 (Certara, USA), Dose-normalised plasma concentrations of lenalidomide after single subcutaneous administration of the formulation was assessed and the following plasma pharmacokinetic parameters was evaluated:
Cₘₐₓ—The maximum observed plasma concentration (ng/ml)
Tₘₐₓ—Time to reach Cₘₐₓ (h)
AUC₀₋₂₄ₕ—The AUC from time 0 to the time at 24 hours (h·ng/ml).
AUCₗₐₛₜ—The AUC from time 0 to the time of the last detectable or last measured (at 336 h) plasma concentration (h·ng/ml).

Nominal plasma sampling timepoints and nominal doses were used for the non-compartmental PK analysis. PK for animals were calculated with the extravascular dose option within WinNonlin. Reported plasma concentrations below LLOQ but above 3 ng/ml were included in the PK analysis, whereas non-reported plasma concentrations below LLOQ were omitted from analysis.

Cₘₐₓ, and Tₘₐₓ were derived from the observed plasma concentration data. AUC was assessed by integration of the plasma concentration vs time curve using linear interpolation for increasing plasma levels and logarithmic interpolation for decreasing plasma levels (Linear Up Log Down method).

### Results

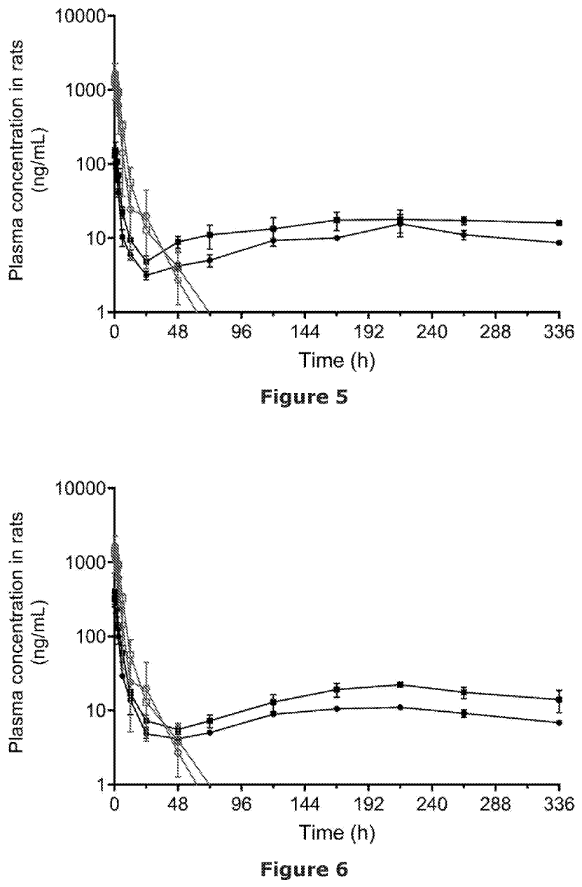

FIG. 5 shows the plasma concentration time curves (ng/ml plasma concentration lenalidomide versus h sampling time) for samples obtained by Example 21 and samples obtained using uncoated microparticles of lenalidomide as a control.

More specifically, the plasma concentration (ng/ml) of lenalidomide over time (h) is shown in a semi-log plot (y is log scale). Squares show results from female rats (n=3), circles show results from male rats (n=3). Open symbols show results from the control (uncoated microparticles of lenalidomide) and closed show the results from coated microparticles of lenalidomide according to the Example 21,

TABLE 11
Results of plasma pharmacokinetic parameters evaluation,
averaged values from both male and female rats (n = 6).

| Parameter | Control | Example 21 |
|---|---|---|
| Tₘₐₓ | 0.5 | 0.8 |
| Cₘₐₓ | 1610 | 152 |
| AUC_{0-24 h} | 5310 | 520 |
| AUCₗₐₛₜ | 5520 | 4330 |

The maximum concentration was lower for Example 21 compared with the control, and was also reached more slowly.

AUC₀₋₂₄ₕ and AUCₗₐₛₜ represent the amount of lenalidomide released after 24 hours and 336 hours respectively. For the control, over 96% of the total amount of lenalidomide released was released in the first 24 hours. In contrast, for Example 21, the amount of lenalidomide released after 24 hours was about 12% of that released after 336 hours and, as can be seen in FIG. 5, the drug release was not yet complete.

Accordingly, Example 21 demonstrated that the coated lenalidomide particles produced by stop-flow ALD showed a release profile with lower initial release than the uncoated lenalidomide particles.

### Example 23

### Coated Lenalidomide Microparticles Using Stop-Flow II

Microparticles of lenalidomide 99.9% (Kyongbo, South Korea) were used as received. The mean diameter of the lenalidomide particles was 4 μm as determined as described in Comparative Example 17 above. The particle size distribution, as determined by laser diffraction, was as follows: D₁₀ 0.6 μm; D₅₀ 3.9 μm and D₉₀ 15.6 μm.

The microparticles were coated essentially as described in Example 18, except that the layers were formed in alternating 4:1 (Zn:Al) and 3:1 (Zn:Al) sets, instead of consistent 3:1 (Zn:Al) sets. Accordingly, sets 1, 3 and 5 each included 25 cycles in total with a ratio of 4 cycles of Zn:1 cycle of Al. Meanwhile, sets 2, 4 and 6 each included 24 cycles in total with a ratio of 3 cycles of Zn:1 cycle of Al.

### Example 24

### Determination of Drug Load

The present Example describes the analysis of the drug load of lenalidomide of the particles obtained in Example 23.

### Material and Method

The material and method was as described in Example 12 except that the particles analysed were those obtained in Example 23, rather than those obtained in Example 11.

### Results

The drug load lenalidomide of the particles according to Example 23 was determined as 75.2%.

### Example 25

### Determination of Coating Integrity

The present Example describes the analysis of the coating integrity of the particles obtained in Example 23,

The coating integrity of the coated lenalidomide was determined as described in Example 13.

### Material and Method

The material and method was as described in Example 13 except that the particles analysed were those obtained in Example 23, rather than those obtained in Example 11.

### Results

The amount of API dissolved in the coating integrity assay of the particles according to the Example was determined as 16.2%.

### Example 26

### Preparation of Formulations

For the present example, formulations of the coated lenalidomide particles as disclosed herein were prepared as described in Example 21 except that particles obtained in Example 23 were used rather than particles obtained in Example 18.

### Example 27

### In Vivo Pharmacokinetic Study

The present Example describes a preclinical pharmacokinetic study of lenalidomide following administration of coated lenalidomide formulations according to Example 26 in male and female Sprague Dawley rats.

### Material and Method

Male and Female Sprague Dawley rats weighing between 260 and 343 g at the day of administration were supplied by Charles River (UK). The duration of the study was 14 days. In each study group of the study, 3 rats of each sex were used. The study groups were the coated microparticle formulation according to the Example, and as control, uncoated microparticles of lenalidomide.

The administration areas were clipped free from hair prior to Injection and the injection site will be marked. A suspension prepared as described in Example 26 with coated lenalidomide particles according to Example 23 were drawn into a 1 mL BD syringe and single, subcutaneous injections (ca. 0.12 mL) was administered through a 23 G needle (BD microlance) into the flank of each rat.

Blood samples (ca 0.2 mL) were be collected from the tail vein into K₂EDTA tubes at the following time-points: 0.25, 0.5, 1, 2, 3, 6, 12, 24, 48, 72, 120, 168, 264, and 336 h post-dose. As soon as practically possible following blood sampling, plasma was separated by centrifugation (1500 g for 10 min at 4° C.), Animals were sacrificed on the last day of the study. Plasma samples were stored in a freezer at −80° C. pending analysis.

To determine the plasma concentration, UPLC-MS/MS was used.

Samples were prepared by pipetting 35 μL of rat plasma into a 384 well plate, adding 35 μL 5% DMSO in acetonitrile, 70 μL of an internal standard working solution using the TECAN EVO Ware liquid handling robot. The 384 well plates were shaken for 15 minutes and centrifuged. All samples were then injected on a UPLC-MS/MS system (Xevo TQ-s micro coupled to an Acquity I-Class UPLC system, Waters, Milford, MA, USA) with the set-up as specified in Table 12 below.

TABLE 12
Parameters for the UPLC-MS/MS-method.

| | |
|---|---|
| Column | Acquity CSH C18 (50 × 2.1 mm, 1.7 μm) |
| Mobile phase A | 0.10% Formic Acid in Water |
| Mobile phase B | ACN:DMSO (95:5) |
| Wash solution | ACN:MeOH (1:1) |

Pharmacokinetic analysis of lenalidomide in plasma was performed according to using non-compartmental analysis (NCA) utilizing the software Phoenix WinNonlin, version 8.3 (Certara, USA), Dose-normalised plasma concentrations of lenalidomide after single subcutaneous administration of the formulation was assessed and the following plasma pharmacokinetic parameters was evaluated:
Cₘₐₓ—The maximum observed plasma concentration (ng/ml)
Tₘₐₓ—Time to reach Cₘₐₓ (h)
AUC₀₋₂₄ₕ—The AUC from time 0 to the time at 24 hours (h·ng/mL).
AUCₗₐₛₜ—The AUC from time 0 to the time of the last detectable or last measured (at 336 h) plasma concentration (h·ng/mL).

Nominal plasma sampling timepoints and nominal doses were used for the non-compartmental PK analysis. PK for animals were calculated with the extravascular dose option within WinNonlin. Reported plasma concentrations below LLOQ but above 3 ng/ml were included in the PK analysis, whereas non-reported plasma concentrations below LLOQ were omitted from analysis.

Cₘₐₓ, and Tₘₐₓ were derived from the observed plasma concentration data. AUC was assessed by integration of the plasma concentration vs time curve using linear interpolation for increasing plasma levels and logarithmic interpolation for decreasing plasma levels (Linear Up Log Down method).

### Results

FIG. 6 shows the plasma concentration time curves (ng/ml plasma concentration lenalidomide versus h sampling time) for samples obtained by Example 26 and samples obtained using uncoated microparticles of lenalidomide as a control.

More specifically, the plasma concentration (ng/ml) of lenalidomide over time (h) is shown in a semi-log plot (y is log scale). Squares show results from female rats (n=3), circles show results from male rats (n=3). Open symbols show results from the control (uncoated microparticles of lenalidomide) and closed show the results from coated microparticles of lenalidomide according to the Example 26,

TABLE 13
Results of plasma pharmacokinetic parameters evaluation
averaged values from both male and female rats (n = 6).

| Parameter | Control | Example 26 |
|---|---|---|
| Tₘₐₓ | 0.5 | 0.5 |
| Cₘₐₓ | 1610 | 392 |
| AUC_{0-24 h} | 5310 | 1217 |
| AUCₗₐₛₜ | 5520 | 4892 |

The maximum concentration was lower for Example 21 compared with the control.

For the control, over 96% of the total amount of lenalidomide released was released in the first 24 hours. In contrast, for Example 26, the amount of lenalidomide released after 24 hours was about 25% of that released after 336 hours and, as can be seen in FIG. 6, the drug release was not yet complete.

Accordingly, Example 26 demonstrated that the coated lenalidomide particles produced by stop-flow ALD showed a release profile with lower initial release than the uncoated lenalidomide particles.

### Example 28

### Coated Liraglutide Microparticles Using Stop-Flow

R&D grade liraglutide (MedChemExpress, New Jersey, US) with a purity of 98.5% and a peptide content of 90.8% was suspended in a solution of 0.1% Span 85 (Sigma-Aldrich, MO, USA) in cyclohexane (Merck, Germany). The particle size distribution was determined by means of laser diffraction (SALD-7500nano (Shimadzu, Japan), 405 nm laser) to be as follows: % D(10): 2.0 μm, % D(50): 7.3 μm, % D(90): 23.8 μm.

The raw material was spray dried in a Mini Spray Drier (B-290, med Dehumidifier B-296 with a Two-fluid nozzle; BÜCHI Labortechnik GmbH, Germany), by dispersing the raw material in purified water (0.8-2 MΩ/cm2) to form a milky white liquid with a liraglutide concentration of 15 wt %. The dispersion was spray dried with an inlet temperature of 115° C., aspiration rate 100% (˜35 m³/h), pump rate 8% (3.8 mL/min), nozzle clean 2, volume flow 35 mm (N₂, ˜600 L/h), which gave an outlet temperature of 73° C. The gross mass yield was approximated to 77.5%. The spray-dried material was assayed using a Nexera UPLC-UV-DAD (Shimadzu, Japan) with a SunShell nC18-WP, 4.6×100 mm, 2.6 μm particle size column (Chromanik Technologies Inc., Japan) and was found to have a 100.1±0.2% (relative) liraglutide content in the spray-dried material. The particle size of the spray-dried material, determined as above, was as follows: % D(10): 1.7 μm, % D(50): 8.2 μm, % D(90): 21.2 μm.

The microparticles were coated essentially as described in steps 1˜4 of Comparative Example 10 except that:
prior to step 1, three cycles of Al were performed;
in step 1, the 3 cycles of Zn+1 cycle of Al were repeated ten times per set (resulting in a total of 40 cycles per set);
in step 3, steps 1-2 were repeated four times, forming a second, third, fourth and fifth layer of mixed oxide with an atomic ratio of approximately 3:1 zinc:aluminium;
in step 4, an additional three cycles of Al were performed to complete the process; and
the ALD-cycle was performed as follows (steps a-d represent the first cycle, subsequent cycles start from step a as specified in step e).
a. Reagent pulse 1:
   i. The valve on the piping between the pump and the ALD reactor was closed.
   ii. The valve on the diethylzinc or trimethylaluminium precursor bottle was opened for 1 s letting evaporated metal containing precursor fill the reaction chamber.
   iii. The valve to the precursor bottle was closed and, before opening to the pump again, the chamber was rested for 30 s (soaking time) to ensure the metal containing precursor vapor react with the hydroxyl groups on the surface of the drug particles.
   iv. The reactor was thereafter pumped for 9 s.
   v. Steps a-d above was repeated 20 times.
b. Purging pulse: The chamber was purged with nitrogen in a continuous flow. Non-reacted reagents and organic gases were removed.
c. Reagent pulse:
   i. The valve on the piping between the pump and the ALD reactor was closed,
   ii. The valve on the water precursor bottle was opened for 1 s letting evaporated water fill the reaction chamber.
   ill. The valve to the water precursor bottle was closed and, before opening the pump valve again, the chamber was rested for 30 s (soaking time) to ensure the water vapor adsorb to the surface of the drug particles, presenting hydroxyl groups on the exterior or the particles.
   iv. The reactor was thereafter pumped for 9 s.
   v. Steps i-iv above were repeated 20 times.
d. Purging pulse: The chamber was purged with nitrogen in a continuous flow. Gaseous water, and organic gases in case this is not the first cycle, was removed.
e. The cycle was repeated from step a.

### Example 29

### Determination of Drug Load

The present Example describes the analysis of the drug load of liraglutide of the particles obtained in Example 28.

### Material and Method

To determine the drug load (i.e. w/w % of liraglutide in the powder), HPLC (Prominence-i, Shimadzu, Japan) equipped with a diode array detector (Shimadzu, Japan) set at 220 nm was employed using a 4.6×150 mm, 2.6 μm particles, PS C18 column (Kinetex, Phenomenex Inc., CA, USA) was used. The materials were dissolved in 2 M phosphoric acid in acetonitrile/water (1:1) and was diluted with 0.1% trifluoroacetic acid acetonitrile/water (1:9) and further analyzed with HPLC (n=3).

The HPLC assay is set-up according to Table 14.

TABLE 14
Parameters for the HPLC-method.

| Column | Kinetex PS C18 4.6 × 150 mm, 2.6 μm | |
|---|---|---|
| | particles, Nanexa column ID K017. | |
| Temperature, column oven | 25° C. | |
| Flow | 1 mL/min | |
| | Time (min) | % Mobile phase B |
| Gradient program | 0-1 | 40 |
| | 1-8 | 86 |
| | 8-8.5 | 40 |
| | 8.5-13 | 40 |

| | |
|---|---|
| Mobile phase A | 90% H2O 10% ACN 0.1% TFA |
| Mobile phase B | 80% ACN 20% H2O 0.1% TFA |
| Wash solution | 80% Acetonitrile in water |
| Run time | 13 min |
| Temperature, autosampler | 20° C. |
| Injection volume | 10 μL |
| UV detection wavelength | 220 nm |

Concentrations of injected samples were calculated automatically by the software supplied by the manufacturer. Drug load was calculated according to the equation below, wherein Cₛₐₘₚₗₑ is the analyzed concentration of liraglutide, Vₜ is the total volume of the sample, and mₛₐₘₚₗₑ is sample mass:

$$Drug Load = 100 \cdot \frac{{C}_{sample} \cdot {V}_{t}}{{m}_{sample}}$$

### Results

The drug load liraglutide of the particles according to Example 28 was determined as 53.1%.

### Example 30

### Determination of Coating Integrity (DMSO)

The present Example describes the analysis of the coating integrity of the particles obtained in Example 28.

To determine coating integrity, a sample of particles obtained in Example 28 was suspended in dimethylsulfoxide (Rathburn, UK) at a concentration of 0.4 mg liraglutide per mL of solvent and rotated on overhead stirrer for up to 72 hours. Intermittent samples were taken, centrifuged at 2697 rcf for 10 minutes, and the supernatant was diluted in Mobile Phase A above before injection into the above HPLC system for quantification.

The concentrations of injected samples were calculated automatically by the software supplied by the manufacturer. The amount of API dissolved in the coating integrity assay was calculated according to the equation below, wherein cₐ is the concentration of API from analysis (mg/ml), Vₜ is the total sample volume (ml), m_{API} is the mass of API weighed in (mg), and drug load is the API content of the coated API (fraction):

$$Amount API dissolved ( \% ) = 100 \cdot ( {c}_{a} \cdot {V}_{t} ) / ( {m}_{API} \cdot drug load )$$

### Results

The amount of API dissolved in the coating integrity assay of the particles according to Example 28 was determined as 3.0% after 3 hours, and 5.2% after 72 hours.

### Example 31

### Preparation of Formulations

The present Example describes the preparation of formulations of the coated lenalidomide particles as disclosed herein.

The powder of coated microparticles of liraglutide according to the Example was mixed together with Hyonate® vet (Boehringer Ingelheim Animal Health, France) as vehicle. Hyonate® vet is a veterinary medicinal product used in injecting animals comprising a sterile, isotonic, phosphate buffered solution of 10 mg/ml of sodium hyaluronate (pH 7.4). The composition of Hyonate® vet is presented in Table 15 below.

TABLE 15
Composition of the vehicle for suspension
of particles as disclosed herein.

| | | | |
|---|---|---|---|
| Sodium hyaluronate | 10 | mg | Active substance |
| Sodium chloride | 8.5 | mg | Isotonic agent |
| Sodium acid phosphate | 0.223 | mg | Buffering agent |
| Sodium dihydrogen | 40 | μg | Buffering agent |

| | | |
|---|---|---|
| phosphate monohydrate | | |
| Hydrochloric acid | q.s. | For pH adjustment |
| Sodium hydroxide | q.s. | For pH adjustment |
| Water for injection | q.s. ad 1 mL | Solvent |

The microparticles were reconstituted by adding the vehicle to a vial with powder of microparticles to obtain a liraglutide concentration of:
10 mg/ml liraglutide of the particles according to the Example 28.

### Example 32

### In Vivo Pharmacokinetic Study

The present Example describes a preclinical pharmacokinetic study of liraglutide following administration of coated liraglutide formulations according to Example 31 in male Sprague Dawley rats.

### Material and Method

Male Sprague Dawley rats weighing approximately 300 g at the day of administration were supplied by Charles River (UK). The duration of the study was 28 days. In each study group of the study, 4 rats were used. The study groups were the coated microparticle formulation according to Example 31, and as control, liraglutide solution (Victoza®, Novo Nordisk, Denmark) diluted 10× in normal saline for injection.

The administration areas were clipped free from hair prior to injection and the injection site will be marked. A suspension prepared as described in Example 32 with coated liraglutide particles according to Example 28 were drawn into a 1 mL BD syringe and single, subcutaneous injections (ca. 0.08 mL and 0.17 mL) was administered through a 23 G needle (BD Microlance) into the flank of each rat. In the control group, the control preparation (ca. 0.10 mL) was injected intravenously in the tail vein, and following a 7-day wash out period, subcutaneously in the flank of each rat.

Blood samples (ca 0.2 mL) were collected from the jugular vein into K₂EDTA tubes at the following time-points: 1, 3, 6, 12, 24, 48, 72, 120, 168, 251, 384, 480, 576 and 672 hours post-dose from animals who had been administered the coated microparticles according to the Example. Blood samples were collected at the following time-points: 0.25, 1, 3, 6, 9, 12 and 24 hours post-dose following intravenous injection of the control preparation, and at the following time-points: 1, 2, 3, 6, 9, 12, 24, and 48 hours following subcutaneous injection of the control preparation.

As soon as practically possible following blood sampling, plasma was separated by centrifugation (1500 g for 10 min at 4° C.). Animals were sacrificed on the last day of the study. Plasma samples were stored in a freezer at −80° C. pending analysis.

To determine the plasma concentration, UPLC-MS/MS was used.

Samples were prepared by pipetting 35 μL of rat plasma into a 384 well plate, adding 35 μL 5% DMSO in acetonitrile, 70 μL of an internal standard working solution using the TECAN EVO-2 liquid handling robot. The 384 well plates were shaken for 15 minutes and centrifuged. All samples were then injected on a UPLC-MS/MS system (Xevo TQ-s micro coupled to an Acquity I-Class UPLC system, Waters, MA, USA) with the set-up as specified in Table 16 below.

TABLE 16
Parameters for the UPLC-MS/MS-method.

| | |
|---|---|
| Column | ACQUITY UPLC Protein BEH C4 Column, 300 Å, |
| | 1.7 μm, 2.1 mm × 50 mm (Waters, MA, USA) |
| Column | 60° C. |
| temperature | |
| Mobile phase A | 0.30% Formic Acid in Water |
| Mobile phase B | Acetonitrile |
| Wash solution | 1.0% Formic Acid in Methanol |

Pharmacokinetic analysis of lenalidomide in plasma was performed according to using non-compartmental analysis (NCA) utilizing the software Phoenix WinNonlin, version 8.3 (Certara, USA). Dose-normalised plasma concentrations of lenalidomide after single subcutaneous administration of the formulation was assessed and the following plasma pharmacokinetic parameters was evaluated:
Cₘₐₓ—The maximum observed plasma concentration (ng/ml)
tₘₐₓ—Time to reach Cₘₐₓ(h)
tₗₐₛₜ—The time of the last detectable or last measured (at 672 h) plasma concentration (h).
AUC₀₋₂₄ₕ—The area under the curve (AUC) from time 0 to the time at 24 hours (h·ng/ml).
AUCₗₐₛₜ—The AUC from time 0 to fast (h·ng/ml).
t_{1/2,z}—The terminal half-life (h)
AUC_{inf}—AUC extrapolated to infinity (h·ng/ml).
F_{abs}—The absolute bioavailability.
Fᵣₑₗ—The relative bioavailability.
R₀₋₂₄ₕ—The fraction released after 24 hours post injection.

Nominal plasma sampling timepoints and nominal doses were used for the non-compartmental PK analysis. PK for animals were calculated with the extravascular dose option within WinNonlin. Reported plasma concentrations below LLOQ but above 1 ng/ml were included in the PK analysis, whereas non-reported plasma concentrations below LLOQ were omitted from analysis.

Cₘₐₓ, and tₘₐₓ were derived from the observed plasma concentration data. AUC was assessed by integration of the plasma concentration vs time curve using linear Interpolation for increasing plasma levels and logarithmic interpolation for decreasing plasma levels (Linear Up Log Down method). For AUC_{inf}, the area was extrapolated from AUCₗₐₛₜ to infinity using the concentration in the last quantifiable sample and λ_{z}, the first order rate constant associated with the terminal portion of the curve. t_{1/2,z} was calculated by ln 2/λ_{z}. F_{abs} was calculated as a fraction of the AUC_{inf} from intravenous injection and subcutaneous injection, normalized by dose. Fᵣₑₗ was calculated as a fraction of AUC_{inf} from subcutaneous administration of the control formulation and the coated microparticle administration according to Example 31, normalized by dose.

### Results

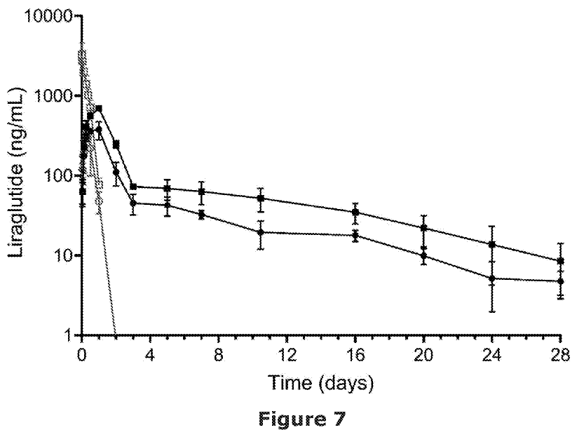

FIG. 7 shows the plasma concentration time curves (ng/ml plasma concentration liraglutide versus days sampling time) for samples obtained by the Example 31 and samples obtained using the known liraglutide solution as a control.

More specifically, plasma concentration (ng/ml) over time (days) is shown in a semi-log plot (y is log scale). Filled squares show results from rats who were administered the coated microparticles of lenalidomide according to the Example at 5.6 mg/kg (n=4). Filled circles show results from rats who were administered the coated microparticles of lenalidomide according to the Example at 2.8 mg/kg (n=4). Open squares show results from rats who were administered the control (liraglutide solution) intravenously at 0.2 mg/kg. Open circles show results from rats who were administered the control (liraglutide solution) subcutaneously at 0.2 mg/kg.

TABLE 17
Results of plasma pharmacokinetic parameters evaluation.

| | Control | Control | Example | Example |
|---|---|---|---|---|
| Parameter | (i.v.) | (s.c.) | (s.c.) | (s.c.) |
| Dose | 0.2 | 0.2 | 2.8 | 5.6 |
| tₘₐₓ | — | 6 | 18 | 21 |
| Cₘₐₓ | 3 433 | 343 | 391 | 670 |
| tₗₐₛₜ | 24 | 48 | 672 | 672 |
| t_{1/2, z} | 4.6 | 4.4 | 142 | 153 |
| AUC_{0-24 h} | — | — | 7 403 | 11 250 |
| AUC_{0-72 h} | — | — | 13 350 | 24 475 |
| AUCₗₐₛₜ | 21 067 | 4 616 | 25 310 | 46 500 |
| AUC_{inf} | 21 230 | 4 622 | 26 310 | 48 775 |
| F_{abs} | 1 | 0.2 | 0.1 | 0.1 |
| Fᵣₑₗ | 5.1 | 1 | 0.4 | 0.4 |
| R_{0-24 h} | — | — | 0.3 | 0.2 |

### Example 33

Injectability 100 mg/mL i Hyonate

### Preparation of Formulations

The powder of coated microparticles of liraglutide according to the Example 28 was mixed together with Hyonate® vet (Boehringer Ingelheim Animal Health, France) as vehicle.

The microparticles were reconstituted by adding the vehicle to a vial with powder of microparticles to obtain a liraglutide concentration of:
100 mg/ml liraglutide of the particles according to the Example 28.

### Material and Method

A suspension prepared as described above with coated liraglutide particles according to Example 28 were drawn into a 1 mL HSW HENKE-JECT® three-part syringe (Henke-Sass, Wolf GmbH, Germany). The syringe was fitted with a 27 G×19 mm injection needle (Microlance 3, BD, USA) and mounted in a custom-built syringe holder inside a TA.XTplus Texture Analyser instrument with Exponent Connect software (Stable Microsystems Ltd., UK). The texture analyser was used to measure the force required to extend a piston while depressing syringe piston to dispense the contents. In the method, the instrument was operated in compression test mode, moving its piston with a test speed of 10 mm/s until a threshold force of 50 N was measured, corresponding to full dispensing of the syringe content into air. A threshold of 15 N was considered as the upper threshold for acceptable force during injection.

The maximum injection force of the suspension described above was determined as 10.3 N.

1. A process for the preparation of pharmaceutical or veterinary composition in the form of a plurality of particles, which process comprises:
(a) loading a plurality of solid cores comprising a biologically-active agent into a stationary gas phase deposition reactor chamber; and
(b) applying a gas phase deposition technique to surround, enclose and/or encapsulate said cores with one or more layers comprising one or more coating materials, each comprising one or more metal-containing or metalloid-containing compounds; and
(c) sequentially repeating step (b) above as required to form a plurality of particles having a weight-, number-, and/or volume-based mean diameter that is between about 10 nm and about 100 μm, each particle comprising a respective solid core and a coating surrounding, enclosing and/or encapsulating said core,
which gas phase deposition technique comprises:
(1) introducing a pulse of a first reactant gas into the stationary gas phase deposition reactor chamber and allowing the first reactant gas to contact said solid cores for a pre-determined period of soaking time;
(2) after step (1), evacuating and/or purging with an inert gas the stationary gas phase deposition reactor chamber;
(3) introducing a pulse of a second reactant gas into the stationary gas phase deposition reactor chamber and allowing the second reactant gas to contact said solid cores for a pre-determined period of soaking time; and
(4) after step (3), evacuating and/or purging with an inert gas the stationary gas phase deposition reactor chamber,
wherein either the first reactant gas or the second reactant gas comprises a metal or metalloid-containing compound.

2. The process as claimed in claim 1, wherein step (1), step (3) or both steps (1) and (3) comprise allowing the respective reactant gas to contact said solid cores for the respective pre-determined period of soaking time in the substantial absence of pumping that may result in flow of gases.

3. The process as claimed in claim 1, wherein step (1), step (3) or both steps (1) and (3) comprise allowing the respective reactant gas to contact said solid cores for the respective pre-determined period of soaking time in the substantial absence of mechanical agitation of the plurality of solid cores.

4. The process as claimed in claim 1, wherein the pre-determined period of soaking time is from about 2 seconds to about 30 minutes.

5. The process as claimed in claim 1, wherein step (1), step (3) or both steps (1) and (3) comprise:
a. after said pre-determined period of soaking time, pumping the reactor for a pre-determined period of pumping time; and
b. repeating, a pre-determined number of times, the steps of introducing a pulse of the respective reactant gas, allowing the respective reactant gas to contact said solid cores for a pre-determined period of soaking time, and pumping the reactor for a pre-determined period of pumping time.

6. The process as claimed in claim 1, wherein each repetition of step (b) is carried out using the same, or different, first and second reactant gases to the previous iteration of step (b).

7. The process as claimed in claim 1, wherein, between one or more sequential pairs of step (b) iterations, the process comprises deagglomerating the coated solid cores.

8. The process as claimed in claim 7, wherein one or more instances of deagglomerating the coated solid cores comprises removing the coated solid cores from the stationary gas phase deposition reactor chamber, vibrational sieving or sonic sifting the coated solid cores and re-loading the coated solid cores into the stationary gas phase deposition reactor chamber in order to repeat step (b).

9. The process as claimed in claim 8, wherein vibrational sieving or sonic sifting of the coated solid cores is carried out using a sieve with a mesh size determined so that the ratio of the size of sieved or sifted particles to the sieve mesh size is about 1:>1, preferably about 1:2, and optionally is about 1:4.

10. The process as claimed in claim 1, wherein at least 200 mg of the solid cores are loaded into the stationary gas phase deposition reactor chamber for the gas phase deposition technique to be applied, optionally at least 1 g or at least 10 g.

11. The process as claimed in claim 1, wherein between 3 and 10 discrete layers of coating material are applied to the core sequentially.

12. The process as claimed in claim 1, wherein, the total thickness of the discrete layers of coating material is between about 0.5 nm and about 2 μm.

13. The process as claimed in claim 1, wherein the maximum thickness of an individual discrete layer of coating material is about 1 hundredth of the weight-, number-, or volume-based mean diameter of the core, including any other previously-applied discrete layers of coating material that are located between said individual discrete layer and the outer surface of the core.

14. The process as claimed in claim 1, wherein the coating materials of the one or more discrete layers comprise one or more inorganic coating materials.

15. The process as claimed in claim 14, wherein the one or more metal-containing, or metalloid-containing, compounds comprise a hydroxide and/or an oxide.

16. The process as claimed in claim 14, wherein the one or more coating materials comprise silicon oxide, aluminium oxide, titanium dioxide, zinc sulphide and/or zinc oxide.

17. The process as claimed in claim 16, wherein the one or more coating materials comprise a mixture of zinc oxide along with one or other or both of silicon dioxide and aluminium oxide.

18. The process as claimed in claim 1, which comprises applying the separate layers of coating materials to cores, and/or previously-coated cores, by atomic layer deposition.

19. The process as claimed in claim 1, wherein the cores comprise a pharmaceutically-acceptable excipient.

20. The process as claimed in claim 1, wherein the carrier/excipient material is a sugar or a sugar alcohol and/or is a pH modifying agent.

21. The process as claimed in claim 1, wherein the cores consist essentially of biologically active agent.

22. The process as claimed in claim 1, wherein the biologically active agent is selected from an analgesic, an anaesthetic, an anti-ADHD agent, an anorectic agent, an antiaddictive agent, an antibacterial agent, an antimicrobial agent, an antifungal agent, an antiviral agent, an antiparasitic agent, an antiprotozoal agent, an anthelmintic, an ectoparasiticide, a vaccine, an anticancer agent, an antimetabolite, an alkylating agent, an antineoplastic agent, a topoisomerase, an immunomodulator, an immunostimulant, an immunosuppressant, an anabolic steroid, an anticoagulant agent, an antiplatelet agent, an anticonvulsant agent, an antidementia agent, an antidepressant agent, an antidote, an antihyperlipidemic agent, an antigout agent, an antimalarial, an antimigraine agent, an anti-inflammatory agent, an antiparkinson agent, an antipruritic agent, an antipsoriatic agent, an antiemetic, an anti-obesity agent, an anthelmintic, an antiasthma agent, an antibiotic, an antidiabetic agent, an antiepileptic, an antifibrinolytic agent, an antihemorrhagic agent, an antihistamine, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, a antidiarrheals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radio-pharmaceutical, a hormone, a sedative, an anti-allergic agent, an appetite stimulant, a steroid, a sympathomimetic, a thrombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a cytokine, a peptidomimetic, a peptide, a protein, a radiopharmaceutical, a senotherapeutic, a toxoid, a serum, an antibody, a nucleoside, a nucleotide, a vitamin, a portion of genetic material, a nucleic acid, or a mixture of any two or more of said biologically active agents.

23. The process as claimed in claim 1, wherein the biologically-active agent is an anti-cancer agent.

24. The process as claimed in claim 23, wherein the biologically-active agent is azacitidine.

25. A composition obtained by the process as claimed in claim 1.

26. A pharmaceutical or veterinary formulation comprising a composition as claimed in claim 25 and a pharmaceutically-acceptable or a veterinarily-acceptable adjuvant, diluent or carrier.

27. The pharmaceutical or veterinary formulation as claimed in claim 26 in the form of a sterile injectable and/or infusible dosage form.

28. The pharmaceutical or veterinary formulation as claimed in claim 26 in the form of a liquid, a sol or a gel, administrable via a surgical administration apparatus that forms a depot formulation.

29. A process for the preparation of a pharmaceutical or veterinary formulation, which comprises admixing a composition as defined in claim 25 with the relevant pharmaceutically-acceptable or a veterinarily-acceptable adjuvant, diluent or carrier.

30. The composition as claimed in claim 25, in which the biologically active agent is an anti-cancer agent.

31. (canceled)

32. A method of treatment of cancer, which method comprises administration of the composition as claimed in claim 30 to patient in need of such treatment.

33. The method as claimed in claim 32, wherein the anti-cancer agent is azacitidine and the cancer is myelodysplastic syndrome or one or more of its sub-types.

34. The composition as claimed in claim 25 in which the biologically active agent is selected from an analgesic, an anaesthetic, an anti-ADHD agent, an anorectic agent, an antiaddictive agent, an antibacterial agent, an antimicrobial agent, an antifungal agent, an antiviral agent, an antiparasitic agent, an antiprotozoal agent, an anthelminic, an ectoparasiticide, a vaccine, an anticancer agent, an antimetabolite, an alkylating agent, an antineoplastic agent, a topoisomerase, an immunomodulator, an immunostimulant, an immunosuppressant, an anabolic steroid, an anticoagulant agent, an antiplatelet agent, an anticonvulsant agent, an antidementia agent, an antidepressant agent, an antidote, an antihyperlipidemic agent, an antigout agent, an antimalarial, an antimigraine agent, an anti-inflammatory agent, an antiparkinson agent, an antipruritic agent, an antipsoriatic agent, an antiemetic, an anti-obesity agent, an anthelmintic, an antiasthma agent, an antibiotic, an antidiabetic agent, an antiepileptic, an antifibrinolytic agent, an antihemorrhagic agent, an antihistamine, an antitussive, an antihypertensive agent, an antimuscarinic agent, an antimycobacterial agent, an antioxidant agent, an antipsychotic agent, an antipyretic, an antirheumatic agent, an antiarrhythmic agent, an anxiolytic agent, an aphrodisiac, a cardiac glycoside, a cardiac stimulant, an entheogen, an entactogen, an euphoriant, an orexigenic, an antithyroid agent, an anxiolytic sedative, a hypnotic, a neuroleptic, an astringent, a bacteriostatic agent, a beta blocker, a calcium channel blocker, an ACE inhibitor, an angiotensin II receptor antagonist, a renin inhibitor, a beta-adrenoceptor blocking agent, a blood product, a blood substitute, a bronchodilator, a cardiac inotropic agent, a chemotherapeutic, a coagulant, a corticosteroid, a cough suppressant, a diuretic, a deliriant, an expectorant, a fertility agent, a sex hormone, a mood stabilizer, a mucolytic, a neuroprotective, a nootropic, a neurotoxin, a dopaminergic, a free radical scavenging agent, a growth factor, a fibrate, a bile acid sequestrants, a cicatrizant, a glucocorticoid, a mineralcorticoid, a haemostatic, a hallucinogen, a hypothalamic-pituitary hormone, an immunological agent, a laxative agent, a antidiarrheals agent, a lipid regulating agent, a muscle relaxant, a parasympathomimetic, a parathyroid calcitonin, a serenic, a statin, a stimulant, a wakefulness-promoting agent, a decongestant, a dietary mineral, a biphosphonate, a cough medicine, an ophthamological, an ontological, a H1 antagonist, a H2 antagonist, a proton pump inhibitor, a prostaglandin, a radio-pharmaceutical, a hormone, a sedative, an anti-allergic agent, an appetite stimulant, a steroid, a sympathomimetic, a thrombolytic, a thyroid agent, a vasodilator, a xanthine, an erectile dysfunction improvement agent, a gastrointestinal agent, a histamine receptor antagonist, a keratolytic, an antianginal agent, a non-steroidal antiinflammatory agent, a COX-2 inhibitor, a leukotriene inhibitor, a macrolide, a NSAID, a nutritional agent, an opioid analgesic, an opioid antagonist, a potassium channel activator, a protease inhibitor, an antiosteoporosis agent, a cognition enhancer, an antiurinary incontinence agent, a nutritional oil, an antibenign prostate hypertrophy agent, an essential fatty acid, a non-essential fatty acid, a cytokine, a peptidomimetic, a peptide, a protein, a radiopharmaceutical, a senotherapeutic, a toxoid, a serum, an antibody, a nucleoside, a nucleotide, a vitamin, a portion of genetic material, a nucleic acid, or a mixture of any two or more of said biologically active agents.
